SEQUENCE LISTING (70 sequences)

SEQ ID NO: 1 - protein, 12 aa, Artificial Sequence
  Anionic spacer
AEAEPEAEAEGK

SEQ ID NO: 2 - protein, 12 aa, Artificial Sequence
  Anionic spacer
AEAEPEAEAAGK

SEQ ID NO: 3 - protein, 13 aa, Artificial Sequence
  Anionic spacer
AEAEPEAEAEGPK

SEQ ID NO: 4 - protein, 13 aa, Artificial Sequence
  Anionic spacer
AEAEPEAEAAGPK

SEQ ID NO: 5 - protein, 13 aa, Artificial Sequence
  Anionic spacer
AEAEPELAEAAGK

SEQ ID NO: 6 - protein, 13 aa, Artificial Sequence
  Anionic spacer
AEAEPELVEAAGK

SEQ ID NO: 7 - protein, 13 aa, Artificial Sequence
  Anionic spacer
MAEAEPEAEPIMK

SEQ ID NO: 8 - protein, 14 aa, Artificial Sequence
  Anionic spacer
MAEAEPEAEEPIMK

SEQ ID NO: 9 - protein, 14 aa, Artificial Sequence
  Anionic spacer
MAEAEPEAEAPIMK

SEQ ID NO: 10 - protein, 14 aa, Artificial Sequence
  Anionic spacer
MAEAEPEAEPIMEK

SEQ ID NO: 11 - protein, 14 aa, Artificial Sequence
  Anionic spacer
MAEAEPEAEPIMVK

SEQ ID NO: 12 - protein, 12 aa, Artificial Sequence
  Indolicidin analogue 11B7
ILRWPWWPWRRK

SEQ ID NO: 13 - protein, 13 aa, Artificial Sequence
  Indolicidin analogue 11F2
ILKKWPWWVWRRK

SEQ ID NO: 14 - protein, 12 aa, Artificial Sequence
  Indolicidin analogue 11F4
ILRWVWWVWRRK

SEQ ID NO: 15 - protein, 13 aa, Artificial Sequence
  Indolicidin analogue 11F5
ILRRWVWWVWRRK

SEQ ID NO: 16 - protein, 12 aa, Artificial Sequence
  Indolicidin analogue 11G6
ILKKWPWWPRRK

SEQ ID NO: 17 - protein, 12 aa, Artificial Sequence
  Indolicidin analogue 11H11
ILRWPWWPWRAK

SEQ ID NO: 18 - protein, 11 aa, Artificial Sequence
  Anionic spacer
HEAEPEAEPIM

SEQ ID NO: 19 - protein, 8 aa, Artificial Sequence
  Anionic spacer
EAEPEAEP

SEQ ID NO: 20 - protein, 8 aa, Artificial Sequence
  Anionic spacer
EAKPEAEP

SEQ ID NO: 21 - protein, 8 aa, Artificial Sequence
  Anionic spacer
EAEPKAEP

SEQ ID NO: 22 - protein, 8 aa, Artificial Sequence
  Anionic spacer
EAESEAEP

SEQ ID NO: 23 - protein, 8 aa, Artificial Sequence
  Anionic spacer
EAELEAEP

SEQ ID NO: 24 - protein, 6 aa, Artificial Sequence
  Anionic spacer
EPEAEP

SEQ ID NO: 25 - protein, 4 aa, Artificial Sequence
  Anionic spacer
EAEP

SEQ ID NO: 26 - protein, 14 aa, Artificial Sequence
  Anionic spacer
MEAEPEAEPIMEKR

SEQ ID NO: 27 - protein, 14 aa, Artificial Sequence
  Anionic spacer
MEAEPEAEPIMVKR

SEQ ID NO: 28 - DNA, 121 bp, Artificial Sequence
  Exemplary “template”
tgctaccacctcaggatccggctccggaagcggaagcagmgggtaaaattctgcgttggccgtggtggccgtggcgtcgcaaagccgaagcggaaccggtgtaataacctcgagggtcgct

SEQ ID NO: 29 - DNA, 123 bp, Artificial Sequence
  Exemplary “template”
tgcttaggatccgagcggtccgaaaattctgcgttggccgtggtggccgtggcgtcgcaaagccgaagcggaaccggaagcggaagcagmgggtccttaataagcttggtaccccgatgcttg

SEQ ID NO: 30 - DNA, 166 bp, Artificial Sequence
  Exemplary “template”
gcggaagcagcgggtaaaattctgcgttggccgtggtggccgtggcgtcgcaaagccgaagcggaaccggtgtaataacctcgagggggggcccggtacccagcttttgttccctttagtgagggttaattgcgcgcttggcgtaatcatggtcatagctgtttcc

SEQ ID NO: 31 - DNA, 139 bp, Artificial Sequence
  Exemplary “template”
cgccagggttttcccagtcacgacggatccgtctcatatgattctgcgttggccgtggtggccgtggcgtcgcaaaatggccgaagcggaaccggaagcggaaccgattaattaagcttcgatcctctacgccggacgc

SEQ ID NO: 32 - DNA, 72 bp, Artificial Sequence
  Exemplary “template”
tatgattctgcgttggccgtggtggccgtggcgtcgcaaaatggccgaagcggaaccggaagcggaaccgat

SEQ ID NO: 33 - protein, 11 aa, Artificial Sequence
  Indolicidin analogue 11B25
ILRWPWWPWRR

SEQ ID NO: 34 - DNA, 23 bp, Artificial Sequence
  PCR primer
tgctaccacctcaggatccggct

SEQ ID NO: 35 - DNA, 22 bp, Artificial Sequence
  PCR primer
agcgaccctcgaggttattaca

SEQ ID NO: 36 - DNA, 21 bp, Artificial Sequence
  PCR primer
cgattggatccgtctcatatg

SEQ ID NO: 37 - DNA, 20 bp, Artificial Sequence
  PCR primer
acggatcgcaagcttactaa

SEQ ID NO: 38 - DNA, 22 bp, Artificial Sequence
  PCR primer
tgcttaggatccgagcggtccg

SEQ ID NO: 39 - DNA, 20 bp, Artificial Sequence
  PCR primer
caagcatcggggtaccaagc

SEQ ID NO: 40 - DNA, 36 bp, Artificial Sequence
  PCR primer
cactctccggaactggyggaagcagcgggtaaaatt

SEQ ID NO: 41 - DNA, 19 bp, Artificial Sequence
  PCR primer
ggaaacagctatgaccatg

SEQ ID NO: 42 - DNA, 34 bp, Artificial Sequence
  PCR primer
actcaccatatgaaaattctgcgttggccgtggt

SEQ ID NO: 43 - DNA, 20 bp, Artificial Sequence
  PCR primer
gcgtccggcgtagaggatcg

SEQ ID NO: 44 - DNA, 41 bp, Artificial Sequence
  PCR primer
ttcaaagcttaattaatcggtkcttccgcttccggttccgc

SEQ ID NO: 45 - DNA, 37 bp, Artificial Sequence
  PCR primer
actcaccatatggwgaaaattctgcgttggccgtggt

SEQ ID NO: 46 - DNA, 34 bp, Artificial Sequence
  PCR primer
actcaccatatgaagattctgcgttggccgtggt

SEQ ID NO: 47 - protein, 24 aa, Artificial Sequence
  Indolicidin analogue/anionic spacer fusion.
ILRWPWWPWRRKAEAEPEAEAEGK

SEQ ID NO: 48 - protein, 24 aa, Artificial Sequence
  Indolicidin analogue/anionic spacer fusion.
ILRWPWWPWRRKAEAEPEAEAAGK

SEQ ID NO: 49 - protein, 25 aa, Artificial Sequence
  Indolicidin analogue/anionic spacer fusion.
ILRWPWWPWRRKAEAEPEAEAEGPK

SEQ ID NO: 50 - protein, 25 aa, Artificial Sequence
  Indolicidin analogue/anionic spacer fusion.
ILRWPWWPWRRKAEAEPEAEAAGPK

SEQ ID NO: 51 - protein, 25 aa, Artificial Sequence
  Indolicidin analogue/anionic spacer fusion.
ILRWPWWPWRRKAEAEPELAEAAGK

SEQ ID NO: 52 - protein, 25 aa, Artificial Sequence
  Indolicidin analogue/anionic spacer fusion.
ILRWPWWPWRRKAEAEPELVEAAGK

SEQ ID NO: 53 - protein, 25 aa, Artificial Sequence
  Indolicidin analogue/anionic spacer fusion.
ILRWPWWPWRRKMAEAEPEAEPIMK

SEQ ID NO: 54 - protein, 26 aa, Artificial Sequence
  Indolicidin analogue/anionic spacer fusion.
ILRWPWWPWRRKMAEAEPEAEEPIMK

SEQ ID NO: 55 - protein, 26 aa, Artificial Sequence
  Indolicidin analogue/anionic spacer fusion.
ILRWPWWPWRRKMAEAEPEAEAPIMK

SEQ ID NO: 56 - protein, 26 aa, Artificial Sequence
  Indolicidin analogue/anionic spacer fusion.
ILRWPWWPWRRKMAEAEPEAEPIMEK

SEQ ID NO: 57 - protein, 26 aa, Artificial Sequence
  Indolicidin analogue/anionic spacer fusion.
ILRWPWWPWRRKMAEAEPEAEPIMVK

SEQ ID NO: 58 - protein, 25 aa, Artificial Sequence
  Indolicidin analogue/anionic spacer fusion.
ILRWPWWPWRRMEAEPEAEPIMEKR

SEQ ID NO: 59 - protein, 25 aa, Artificial Sequence
  Indolicidin analogue/anionic spacer fusion.
ILRWPWWPWRRMEAEPEAEPIMVKR

SEQ ID NO: 60 - protein, 12 aa, Artificial Sequence
  Indolicidin analouge 11B7CN
ILRWPWWPWRRK

SEQ ID NO: 61 - protein, 37 aa, Artificial Sequence
  Fusion protein
LPPQDPAPEAEAXGKILRWPWWPWRRKAEAEPVPRGS

SEQ ID NO: 62 - DNA, 121 bp, Artificial Sequence
  Nucleic acid template encoding anti-infective peptide 11B7 and anionic spacer peptide S11 or S12
agcgaccctcgaggttattacaccggttccgcttcggctttgcgacgccacggccaccacggccaacgcagaattttacccmctgcttccgcttccggagccggatcctgaggtggtagca

SEQ ID NO: 63 - protein, 36 aa, Artificial Sequence
  Encoded fusion protein
RLDPSHMEKRILRWPWWPWRRMEAEPEAEPINACDP

SEQ ID NO: 64 - DNA, 114 bp, Artificial Sequence
  Nucleic acid template encoding precursor peptide 11B25 and anionic spacer peptide S21.
cgattggatccgtctcatatggagaacgcattctgcgttggccgtggtggccgtggcgtcgcatggaagcggaaccggaagcggaaccgattaattagtaagcttgcgatccgt

SEQ ID NO: 65 - DNA, 114 bp, Artificial Sequence
  Nucleic acid template encoding precursor peptide 11B25 and anionic spacer peptide S21.
acggatcgcaagcttactaattaatcggttccgcttccggttccgcttccatgcgacgccacggccaccacggccaacgcagaatgcgttctccatatgagacggatccaatcg

SEQ ID NO: 66 - DNA, 142 bp, Artificial Sequence
  Synthesized oligonulceotide PR2 and the PR2 sequence.
gcaaacaagagagctcgcagatctgaattaattccattccgcaagggaataaatatctaacaccgtgcgtgttgactattttacctctggcggtgataatggttgcatgtactaaggaggtcatatgtcagttgaattctac

SEQ ID NO: 67 - DNA, 142 bp, Artificial Sequence
  Synthesized oligonulceotide PR2 and the PR2 sequence.
gtagaattcaactgacatatgacctccttagtacatgcaaccattatcaccgccagaggtaaaatagtcaacacgcacggtgttagatatttattcccttgcggaatggaattaattcagatctgcgagctctcttgtttgc

SEQ ID NO: 68 - protein, 12 aa, Artificial Sequence
  Peptide formed during process for amidation of 11B7
ILRWPWWPWRRK

SEQ ID NO: 69 - protein, 12 aa, Artificial Sequence
  Peptide formed during process for amidation of 11B7
ILRWPWWPWRRK

SEQ ID NO: 70 - protein, 11 aa, Artificial Sequence
  Peptide formed during process for amidation of 11B7
ILRWPWWPWRR

(19) United States
(12) Patent Application Publication
(10) Pub. No.: US 2003/0219854 A1
(43) Pub. Date: Nov. 27, 2003

(54) Methods for producing modified anti-infective peptides

(75) Inventors: Maria Marta Guarna, Vancouver (CA); Yuchen Chen, Vancouver (CA); Robert Cory, Vancouver (CA); Jacqui Brinkman, Vancouver (CA); Jennifer Cabralda, North Vancouver (CA); Luba Metlitskaia, North Vancouver (CA); Dinar Suleman, Burnaby (CA)

Correspondence Address: SEED INTELLECTUAL PROPERTY LAW GROUP PLLC, 701 FIFTH AVE, SUITE 6300, SEATTLE, WA, 98104-7092, US

(73) Assignee: MICROLOGIX BIOTECH INC., Vancouver (CA)

(21) Appl. No.: 10/395,896

(22) Filed: Mar. 21, 2003

Related U.S. Application Data
(60) Provisional application No. 60/367,061, filed on Mar. 21, 2002.
(60) Provisional application No. 60/410,711, filed on Sep. 13, 2002.

(51) Int. Cl. C12P 21/06; C07H 21/04; C07K 14/47; C12N 5/06

(52) U.S. Cl. 435/68.1; 435/69.7; 435/320.1; 435/325; 530/350; 536/23.1

(57) ABSTRACT

Compositions and methods for making and using modified anti-infective peptides are provided. For example, synthetically and/or recombinantly produced analogues or derivatives of naturally occurring anti-infective peptides may be efficiently modified using the compositions and methods provided herein to generate similar or identical post-translational modifications found in wild-type anti-infective peptides. The modified anti-infective peptides (e.g., antimicrobial cationic peptides) and analogues or derivatives thereof may be used, for example, in the treatment of microorganism-caused infections.

### BACKGROUND OF THE INVENTION

Anti-infective peptides, particularly cationic peptides, have received increasing attention as a new pharmaceutical substance because of their broad spectrum of antimicrobial activities, which may be helpful to combat the rapid development of multi-drug resistant pathogenic microorganisms. Rapid induction of de novo synthesis and release from storage sites indicates that cationic peptides are involved in the initial host response to microbial infection. An important part of the biosynthesis of many of these anti-infective peptides includes post-translational modification, which may affect peptide function or stability (see generally, e.g., Boman, Immunol Rev 173:5, 2000).

Natural cationic peptides may be isolated from a desired tissue for therapeutic use (Hancock and Lehrer, TIBTECH 16:82, 1998; Gough et al., Infect. Immun. 64:4922, 1996; Steinberg et al., Antimicrob. Agents Chemother. 41:1738, 1997; and Ahmad et al., Biochim. Biophys. Acta 1237:109, 1995). However, the isolation of cationic peptides from natural sources is typically not cost-effective and results in low yields.

Alternatively, these peptides may be synthetically or recombinantly produced. Chemical peptide synthesis can be used to manufacture large amounts, but this approach is also very costly. The use of recombinant synthesis is, in principle, straightforward, but any amino acid sequence that interferes with host growth, such as lytic cationic peptides, is problematic for cloning. Recombinant technology allows the synthesis of larger amounts of a desired protein. However, one limitation is that additional modifications, other than altering amino acid sequence, are not possible by recombinant expression technology.

There have been attempts to chemically modify recombinant proteins (e.g., amidate the carboxy-terminal acid of a protein) by either using in vivo enzymatic modification or in vitro non-enzymatic chemical modifications (see, e.g., U.S. Pat. No. 5,589,346; U.S. Pat. No. 5,503,989; and WO 99/65931). Nonetheless, while chemical transformations of carboxylic acids to carboxamides are known, the reagents usually involved may destroy the sensitive protein backbone. Furthermore, these techniques often are unpredictable and substrate specific, costly and time consuming, require multiple reaction steps to complete the modification, and require a further step of purifying the modified protein.

Accordingly, there is a need for simpler, less harsh, and more versatile methods and compositions to efficiently and economically produce, on a large scale, modified anti-infective peptides. The present invention fulfills this need and, further, provides other related advantages.

### BRIEF SUMMARY OF THE INVENTION

Briefly stated, the present invention provides methods and compositions for combining recombinant and chemical synthetic production of bioactive agents that have incorporated specific post-translational modifications. In one embodiment, a method for producing an anti-infective cationic peptide is provided, comprising contacting a recombinant precursor cationic peptide with at least one amino acid under conditions and for a time sufficient to couple the precursor peptide with said at least one amino acid, wherein said at least one amino acid is a non-natural amino acid, and thereby producing an anti-infective cationic peptide. In one embodiment, the non-natural amino acid is amidated.

In another embodiment, a method for producing an amidated recombinant anti-infective cationic peptide is provided, comprising chemically protecting the carboxy terminus of a recombinant precursor cationic peptide, contacting the protected recombinant precursor cationic peptide with ammonia under conditions and for a time sufficient to amidate the precursor peptide, deprotecting the amidated peptide, and thereby producing an amidated recombinant anti-infective cationic peptide.

In another embodiment, there is provided a method for producing a modified anti-infective cationic peptide, comprising expressing a fusion protein from a nucleic acid expression construct having an expression control element operably linked to a nucleic acid encoding a fusion protein comprising a precursor cationic peptide fused to an anionic spacer, wherein the fusion protein has the structure [(cationic peptide)(cleavage site)(anionic spacer)(cleavage site)]n, wherein n is 5-10; contacting the fusion protein with a cleaving agent to release the precursor cationic peptide from the anionic spacer, wherein the cleaving agent is lysyl endopeptidase; isolating the precursor cationic peptide from the anionic spacer; and contacting the isolated precursor cationic peptide with at least one amino acid under conditions and for a time sufficient to couple the precursor peptide with said at least one amino acid, wherein said at least one amino acid is a non-natural amino acid, and thereby producing a modified anti-infective cationic peptide. In certain embodiments, the precursor cationic peptide is indolicidin analogue 11B25. In other embodiments, the anionic spacer is selected from the group consisting of AEAEPEAEAEGK, AEAEPEAEAAGK, AEAEPEAEAEGPK, AEAEPEAEAAGPK, AEAEPELAEAAGK, AEAEPELVEAAGK, MAEAEPEAEPIMK. MAEAEPEAEEPIMK, MAEAEPEAEAPIMK, MAEAEPEAEPIMEK, or MAEAEPEAEPIMVK. In still other embodiments, the added non-natural amino acid is an amidated natural amino acid, such as a lysine. In other embodiments, the modified cationic peptide is indolicidin analogue 11B7CN.

In further embodiments, the invention provides a method for producing an amidated anti-infective cationic peptide, comprising expressing a fusion protein from a nucleic acid expression construct having an expression control element operably linked to a nucleic acid encoding a fusion protein comprising a precursor cationic peptide fused to an anionic spacer, wherein the fusion protein has the structure [(cationic peptide)(cleavage site)(anionic spacer)(cleavage site)]n, wherein n is 5-10 and the fusion protein is expressed as an inclusion body; solubilizing the fusion protein with ammonium carbonate; contacting the fusion protein with a cleaving agent to release the precursor cationic peptide from the anionic spacer; isolating the precursor cationic peptide from the anionic spacer; and amidating the isolated precursor cationic peptide, and thereby producing an amidated anti-infective cationic peptide. In certain embodiments, the cleaving agent is lysyl endopeptidase. In some embodiments, the anionic spacer is selected from the group consisting of AEAEPEAEAEGK, AEAEPEAEAAGK, AEAEPEAEAEGPK, AEAEPEAEAAGPK, AEAEPELAEAAGK, AEAEPELVEAAGK, MAEAEPEAEPIMK, MAEAEPEAEEPIMK, MAEAEPEAEAPIMK, MAEAEPEAEPIMEK, or MAEAEPEAEPIMVK. In related embodiments, the ammonium carbonate is at a concentration ranging from about 50 mM to about 250 mM. According to any of the aforementioned embodiment, the amidated cationic peptide is indolicidin-analogue 11B7CN.

In still another embodiment, a method for producing an anti-infective cationic peptide is provided, comprising growing a host cell comprising a nucleic acid expression construct having an expression element operably linked to a nucleic acid encoding a fusion protein comprising a precursor cationic peptide fused to an anionic spacer, under conditions to allow expression of the fusion protein; contacting the fusion protein with a cleaving agent to release the precursor cationic peptide from the anionic spacer; isolating the precursor cationic peptide; and contacting the isolated precursor cationic peptide with at least one amino acid under conditions and for a time sufficient to couple the precursor peptide with said at least one amino acid, wherein said at least one amino acid is a non-natural amino acid, and thereby producing an anti-infective cationic peptide. In one embodiment, the non-natural amino acid is amidated.

In a related embodiment, the isolated precursor cationic peptide cleaved from the fusion protein produced in a host cell is protected, the protected precursor peptide is contacted with ammonia under conditions and for a time sufficient to amidate the protected precursor peptide, the amidated protected precursor peptide is deprotected thereby producing an amidated anti-infective cationic peptide.

### BRIEF DESCRIPTION OF THE DRAWINGS

FIGS. 1A and 1B show a nucleic acid sequence template and encoded fusion protein. FIG. 1A is the nucleic acid template encoding anti-infective peptide 11B7 (bold) and anionic spacer peptide S11 or S12 (spacers underlined). Also shown is primer FOR 11B7-S11 for the sense strand (SENSE PRM) and primer REV 11B7-S11 for the antisense strand (ANTISENSE PRM). The M in the nucleotide sequence represents an A to C change, which results in a change from GAG (Glu) in Spacer S11 to GCG (Ala) in Spacer S12. FIG. 1B is a diagram representing the PCR product for 11B7-S11/12.

FIGS. 2A and 2B show a nucleic acid sequence template and encoded fusion protein. FIG. 2A is the nucleic acid template encoding precursor peptide 11B25 (bold) and anionic spacer peptide S21 (spacer underlined). Also shown is primer FOR 11B25 for the sense strand (SENSE PRM) and primer REV 11B25 for the antisense strand (ANTISENSE PRM). The [peptide-spacer] 11B25-S22 is identical to [peptide-spacer] 11B25-S21 except for a Glu (GAG) to Val (GTC) change at amino acid position 8 of the construct. FIG. 2B is a diagram representing the PCR product for 11B25-S21.

FIGS. 3A-3D show multimerization cloning of pBCKS-V-1x11B7-S29 and pBCKS-V-1x11B25-S21. In FIGS. 3A and 3C, pBCKS-V-1x11B7-S29 and pBCKS-V-1x11B25-S21, respectively, are digested with BamHI/AseI to obtain the insert (at right) and with BamHI/NdeI to obtain vector. Ligation of the insert to the respective vector results in the joining of the AseI and NdeI ends, which obliterates the recognition sequence for both enzymes. FIGS. 3B and 3D show the final constructs having two copies of the original fragment (a 2x construct) with one NdeI site near the BamHI site and one AseI site near HindIII.

FIGS. 4A and 4B show a diagram of expression vector pET24C96-5x11B7-S29 and pET24C96-5x11B25-S21, respectively, including a blown up view of the BamHI/HindIII fragment used in its construction. The 10x and 15x vectors are identical except for the cassette size.

FIGS. 5A and 5B show a fragment map of synthesized oligonucleotide PR2 and the PR2 sequence. Primer binding sites (PR2-FOR and PR2-REV) are underlined. Sequences derived directly from the phage λ genome are shown in bold.

FIGS. 6A and 6B show the vector map of pBS-PR2 and a pBS-PR2 fragment blown up showing the expression control region in detail.

FIG. 7 shows vector maps of vector pET24a(+) and expression construct pCI2-CBD96.

FIG. 8 shows the vector map of pCIb.

FIG. 9 shows the vector map of pCIPRe.

FIGS. 10A-10C show indolicidin analogue fusion proteins separated on SDS-PAGE gels after expression from various pET24C96 vectors having from 5 to 15 copies of nucleic acid molecule encoding an indolicidin analogue. (A) shows expression patterns from pET24C96-5x11B7-S11 and S12 and pET24C96-10x11B7-S12 vectors. Samples include low molecular weight (LMW) marker (kDa) (lane 1), pre-induced/induced cultures of cells carrying constructs of C96-5x11B7-S11 (lanes 2, 3, 4, 5), C96-5x11B7-S12 (lanes 6, 7, 8, 9), and C96-10x11B7-S12 (lanes 10, 11, 12, 13). (B) shows expression patterns from pET24C96-15x11B7-S11/S12/S13/S14. Samples analyzed include LMW markers (kDa) (lane 1), pre-induced/induced cultures of cells carrying constructs of C96-15x11B7-S 11 (lanes 2, 3), C96-15x11B7-S12 (lanes 4, 5), C96-15x11B7-S13 (lanes 6, 7), and C96-15x11B7-S14 (lanes 8, 9). (C) shows expression patterns from pET24C96-5x, -10x and -15x11B25-S21 and -S22. Samples analyzed include: MW markers (1, 14), not induced/induced cultures of cells carrying constructs of C96-5x11B25-S21 (2/3), C96-5x11B25-S22 (4/5), C96-10x11B25-S21 (6/7), C96-10x11B25-S22 (8/9), C96-15x11B25-S21 (10/11), C96-15x11B25-S21 (12/13). LMW bands represented from top to bottom are: 97.4; 66.2; 45.0; 31.0; 21.5 and 14.4 kDa.

FIGS. 11A-11C show expression from fermentation of pET24C96 constructs. (A) is an SDS-PAGE gel showing shake flask fermentation of pET24C96-15x11B7-S12. Lane 1, 1 hour of induction; Lane 2, 2 hours of induction; Lane 3, 3 hours of induction; and Lane 4 shows LMW markers. (B) shows growth rate of bacteria carrying pET24C96-15x11B25-S21 before and after induction of expression. (C) is an SDS-PAGE gel from the pET24C96-15x11B25-S21 induction.

FIGS. 12A-12D show SDS-PAGE showing solubilization of fusion proteins. C96-15x111B7-S12 in 8M urea pellet (A, lane 1) and supernatant (A, lane 2), in 4M urea pellet (B, lane 5) and supernatant (B, lane 4), and in SDS pellet (C, lane 7) and supernatant (C, lane 8). LMW standard (Da) in lanes 3 (A) and 6 (B); solubilization of C96-15x11B7-S20 in 8.0 M urea (D, lanes 9 and 10) and in 0.1 M NH₄HCO₃ (lanes 11, 12, and 13). The resulting soluble (lane 9, 11, and 12) and insoluble (lanes 10 and 13) materials were analyzed.

FIGS. 13A and 13B show solubilization of C96-15x11B25-S21 containing inclusion bodies in urea (A) and ammonium bicarbonate (B).

FIGS. 14A-14C show an acid-urea polyacrylamide gel showing cleavage of fusion protein using lysyl endopeptidase in 0.1 M NH₄HCO₃ C96-15x11B7-S12 (A, lane 1) and standard (A, lane 2); C96-15x11B7-S20 (B, lane 3); and C96-15x11B7-S30 (C, lane 4) and standard (C, lane 5).

FIG. 15 shows mass spectrophotometry results confirming the 11B7 peptide is a free acid (MW 1781.74).

FIG. 16 shows inefficient cleavage of C96-15x11B25-S21 fusion protein by Kex2 protease.

FIG. 17 shows an acid-urea polyacrylamide gel showing purification of MBI 11B7 on Q-Macro-prep after lysyl endopeptidase cleavage. Flow through peptide (lane 1), impurities eluted with NaOH (lane 2), standard 11B7 (lane 3).

FIG. 18 shows a schematic method for amidation of precursor peptide 11B7.

FIG. 19 shows MALDI-TOF mass spectrometry results confirming indolicidin analogue 11B7 is amidated (11B7CN) (MW 1779).

FIG. 20 shows an analytical RP-HPLC profile of 11B7CN.

FIG. 21 shows an acid-urea polyacrylamide gel with the migration profile of HPLC purified amidated product 11B7CN (lane 1) next to standard 11B7CN (lane 2).

FIGS. 22A and 22B show (A) a schematic of coupling a Lys amide to precursor peptide 11B25 and (B) a migration profile of reagents used and products obtained in the coupling of precursor peptide 11B25 with Lys amide to obtain anti-infective peptide 11B7 amide (11B7CN). (11B7CN): Boc-11B25 (Lane 1); Boc-11B25-(Boc)K-CN (Lane 2); 11B25 initial (Lane 3); 11B7 standard (Lane 4); 11B7CN product, 5 μg (Lane 6); 11B7CN product, 10 μg (Lane 7); 11B25 initial (Lane 8).

FIG. 23 shows a MALDI-TOF mass spectrum of the coupling product, 11B7CN.

### DETAILED DESCRIPTION OF THE INVENTION

According to the present invention, there are provided methods and compositions to combine recombinant and chemical synthesis technologies for the production of anti-infective cationic peptides, and other bioactive peptides, polypeptides, and proteins, that have non-natural amino acids. Use of recombinant technologies is desired for the ease of large-scale production of bioactive agents, such as anti-infective cationic peptides. However, recombinant hosts, such as Escherichia coli, cannot be used to directly produce peptides and proteins containing certain post-translational modifications (such as amidated amino acids and D-amino acids). Moreover, these post-translational modifications are often important for the desired activity or stability of the bioactive agent. The present invention solves this problem by combining the benefits of recombinant technology (lower cost, higher capacity, and easier sequence modification) with the flexibility of chemical synthesis to facilitate the efficient production of modified peptides and proteins with improved activity and stability.

As used herein, the term “about” or “consists essentially of” refers to ±10% of any indicated structure, value, or range. Any numerical ranges recited herein are to be understood to include any integer within the range and, where applicable (e.g., concentrations), fractions thereof, such as one tenth and one hundredth of an integer (unless otherwise indicated).

Suitable anti-infective peptides include, but are not limited to, naturally occurring cationic peptides and derivatives or analogues thereof. A “purified peptide, polypeptide, or protein” is an amino acid sequence that is essentially free from contaminating cellular components, such as carbohydrate, lipid, nucleic acid (DNA or RNA), or other proteinaceous impurities associated with the polypeptide in nature. Preferably, the purified polypeptide is sufficiently free of contaminants for use in the chemical coupling reactions of the instant invention or for therapeutic use at a desired dose. An “isolated peptide, polypeptide, or protein” is an amino acid sequence that is removed from its original environment, such as being separated from some or all of the co-existing materials in a natural environment (e.g., a natural environment may be a cell).

An anti-infective peptide that is cationic includes peptides that typically exhibit a positive charge at a pH ranging from about 3 to about 10, and contain at least one basic amino acid (e.g., arginine, lysine, histidine). In addition, an anti-infective cationic peptide generally comprises an amino acid sequence having a molecular mass of about 0.5 kDa (i.e., approximately five amino acids in length) to about 10 kDa (i.e., approximately 100 amino acids in length), or a molecular mass of any integer, or fraction thereof (including a tenth and one hundredth of an integer), ranging from about 0.5 kDa to about 10 kDa. Preferably, an anti-infective cationic peptide has a molecular mass ranging from about 0.5 kDa to about 5 kDa (i.e., approximately from about 5 amino acids to about 45 amino acids in length), more preferably from about 1 kDa to about 4 kDa (i.e., approximately from about 10 amino acids to about 35 amino acids in length), and most preferably from about 1 kDa to about 2 kDa (i.e., approximately from about 10 amino acids to about 18 amino acids in length). In another preferred embodiment, the anti-infective cationic peptide is part of a larger peptide or polypeptide sequence having, for example, a total of up to 100 amino acids, more preferably up to 50 amino acids, even more preferably up to 35 amino acids, and most preferably up to 15 amino acids. The present invention contemplates an anti-infective cationic peptide having an amino acid sequence of 5 to 100 amino acids, with the number of amino acids making up the peptide sequence comprising any integer in that range. An anti-infective cationic peptide may exhibit antibacterial activity, anti-endotoxin activity, antifungal activity, antiparasite activity, antiviral activity, anticancer activity, anti-inflammatory activity, wound healing activity, and/or synergistic activity with other peptides or antimicrobial compounds, or a combination thereof.

Exemplary anti-infective peptides include, but are not limited to, cationic peptides such as cecropins, normally made by lepidoptera (Steiner et al., Nature 292:246, 1981) and diptera (Merrifield et al., Ciba Found. Symp. 186:5, 1994), by porcine intestine (Lee et al., Proc. Nat'l Acad. Sci. USA 86:9159,1989), by blood cells of a marine protochordate (Zhao et al., FEBS Lett. 412:144, 1997); synthetic analogues of cecropin A, melittin, and cecropin-melittin chimeric peptides (Wade et al., Int. J. Pept. Protein Res. 40:429,1992); cecropin B analogues (Jaynes et al., Plant Sci. 89:43, 1993); chimeric cecropin A/B hybrids (Düring, Mol. Breed. 2:297, 1996); magainins (Zasloff, Proc. Nat'l Acad. Sci USA 84:5449, 1987); cathelin-associated antimicrobial peptides from leukocytes of humans, cattle, pigs, mice, rabbits, and sheep (Zanetti et al., FEBS Lett. 374:1,1995); vertebrate defensins, such as human neutrophil defensins [HNP 1-4]; paneth cell defensins of mouse and human small intestine (Oulette and Selsted, FASEB J. 10:1280,1996; Porter et al., Infect. Immun. 65:2396, 1997); vertebrate β-defensins, such as HBD-1 of human epithelial cells (Zhao et al., FEBS Lett. 368:331,1995); HBD-2 of inflamed human skin (Harder et al., Nature 387:861, 1997); bovine β-defensins (Russell et al., Infect. Immun. 64:1565, 1996); plant defensins, such as Rs-AFP1 of radish seeds (Fehlbaum et al., J. Biol. Chem. 269:33159,1994); α- and β-thionins (Stuart et al., Cereal Chem. 19:288,1942; Bohlmann and Apel, Annu. Rev. Physiol. Plant Mol. Biol. 42:227, 1991); γ-thionins (Broekaert et al., Plant Physiol. 108:1353, 1995); the anti-fungal drosomycin (Fehlbaum et al., J. Biol. Chem. 269:33159, 1994); apidaecins, produced by honey bee, bumble bee, cicada killer, hornet, yellow jacket, and wasp (Casteels et al., J. Biol. Chem. 269:26107, 1994; Levashina et al., Eur. J. Biochem. 233:694, 1995); cathelicidins, such as indolicidin and derivatives or analogues thereof from bovine neutrophils (Falla et al., J. Biol. Chem. 277:19298,1996); bacteriocins, such as nisin (Delves-Broughton et al., Antonie van Leeuwenhoek J. Microbiol. 69:193,1996); and the protegrins and tachyplesins, which have antifungal, antibacterial, and antiviral activities (Tamamura et al., Biochim. Biophys. Acta 1163:209, 1993; Aumelas et al., Eur. J. Biochem. 237:575,1996; Iwanga et al., Ciba Found. Symp. 186:160,1994).

In certain embodiments, preferred anti-infective peptides are indolicidin or analogues or derivatives thereof (see, e.g., WO 98/07745 and WO 98/40401). For example, the indolicidin isolated from bovine neutrophils is a 13 amino acid peptide, which is tryptophan-rich and amidated at the carboxy-terminus (see Selsted et al., J. Biol. Chem. 267:4292,1992). As noted above, a preferred indolicidin or analogue or derivative thereof comprises 5 to 45 amino acids, more preferably 7 to 35 amino acids, even more preferably 8 to 25 amino acids, and most preferably 10 to 14 amino acids. In addition, an indolicidin analogue preferably has in a range of about 15% to about 45% tryptophan (W) amino acids, more preferably in a range of about 20% to about 40%, and most preferably in a range of about 25% to about 35%. In certain embodiments, the anti-infective cationic peptide is an indolicidin or an analogue or derivative thereof of up to 35 amino acids, comprising at least one of the following sequences: 11B7 (ILRWPWWPWRRK, SEQ ID NO:), 11F2 (ILKKWPWWVWRRK, SEQ ID NO:), 11F4 (ILRWVWWVWRRK, SEQ ID NO:), 11F5 (ILRRWVWWVWRRK, SEQ ID NO:), 11G6 (ILKKWPWWPRRK, SEQ ID NO:), or 11H11 (ILRWPWWPWRAK, SEQ ID NO:), which may be produced by any one of the methods described herein.

An anti-infective cationic peptide of the present invention may be an analogue or derivative thereof. As used herein, the terms “derivative” and “analogue” when referring to an anti-infective cationic peptide, polypeptide, or fusion protein, refer to any anti-infective cationic peptide, polypeptide, or fusion protein that retain essentially the same (at least 50%, and preferably greater than 70, 80, or 90%) or enhanced biological function or activity as such natural peptide, as noted above. The biological function or activity of such analogues and derivatives can be determined using standard methods (e.g., anti-infective, anti-inflammatory, DNA and/or protein synthesis inhibitor), such as with the assays described herein and known in the art. For example, an analogue or derivative may be a proprotein that can be activated by cleavage, or may be a precursor that can be activated or stabilized by an amino acid modification as described herein, to produce an active anti-infective cationic peptide. Alternatively, an anti-infective peptide and analogues or derivatives thereof can be identified by the ability to specifically bind anti-anti-infective peptide antibodies.

Another example of an analogue or derivative includes an anti-infective cationic peptide that has one or more conservative amino acid substitutions, as compared with the amino acid sequence of a naturally occurring cationic peptide. Among the common amino acids, a “conservative amino acid substitution” is illustrated, for example, by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine, or a combination thereof. Furthermore, an analogue or derivative of a cationic peptide may include, for example, non-protein amino acids, such as precursors of normal amino acids (e.g., homoserine and diaminopimelate), intermediates in catabolic pathways (e.g., pipecolic acid and D-enantiomers of normal amino acids), and amino acid analogues (e.g., azetidine-2-carboxylic acid and canavanine).

Yet other embodiments of analogues or derivatives include an anti-infective cationic peptide that retains at least about 60% identity with the parent molecule (i.e., the “parent” molecule will depend on the starting point, whether the parent is, for example, wild-type indolicidin or an analogue of indolicidin), more preferably at least about 70%, 80%, 90%, and most preferably at least about 95%, or any integer in those ranges. As used herein, “percent identity” or “% identity” is the percentage value returned by comparing the whole of the subject polypeptide, peptide, or analogue or variant thereof sequence to a test sequence using a computer implemented algorithm, typically with default parameters. Sequence comparisons can be performed using any standard software program, such as BLAST, tBLAST, PBLAST, or MegAlign. Still others include those provided in the Lasergene bioinformatics computing suite, which is produced by DNASTAR® (Madison, Wis.). References for algorithms such as ALIGN or BLAST may be found in, for example, Altschul, J. Mol. Biol. 219:555-565, 1991; or Henikoff and Henikoff, Proc. Natl. Acad. Sci. USA 89:10915-10919, 1992. BLAST is available at the NCBI website (www.ncbi.nlm.nih.gov/BLAST). Other methods for comparing multiple nucleotide or amino acid sequences by determining optimal alignment are well known to those of skill in the art (see, e.g., Peruski and Peruski, The Internet and the New Biology: Tools for Genomic and Molecular Research (ASM Press, Inc. 1997); Wu et al. (eds.), “Information Superhighway and Computer Databases of Nucleic Acids and Proteins,” in Methods in Gene Biotechnology, pages 123-151 (CRC Press, Inc. 1997); and Bishop (ed.), Guide to Human Genome Computing, 2nd Edition, Academic Press, Inc., 1998). As used herein, “similarity” between two or more peptides or polypeptides is generally determined by comparing the amino acid sequence of one peptide or polypeptide to the amino acid sequence and conserved amino acid substitutes thereto of a second or more peptide or polypeptide. Further, as is known in the art, a consensus sequence may be determined for a group of analogues or derivatives based on the parent compound amino acid sequence.

As used herein, a “precursor” anti-infective peptide and analogue or derivative thereof may have, for example, one or more deletion, insertion, or modification of any amino acid residue, including the amino- or carboxy-terminal amino acids. Within the scope of this invention, methods are provided to couple non-natural amino acids to precursor anti-infective cationic peptides, such as, for example, one or more amino acids having an acetylated, acylated, acryloylated, alkylated, glycosylated (e.g., glucosylated), PEGylated, myristylated, phosphorylated, sulphated, esterified, amidated, homoserine/homoserine lactone, caprolactam, or a conjugated polyalkylene glycol, or a combination thereof. Also contemplated are less common amino acids such as ornithine, diaminobutyric acid, diaminopropionic acid, D-amino acid, and β-amino acid. Other examples of modified amino acids may include 2,3-diamino butyric acid, 3- or 4-mercaptoproline derivatives, N⁵-acetyl-N⁵-hydroxy-L-ornithine, and α-N-hydroxyamino acids. Additionally, a peptide may be modified to form a polymer-modified peptide. As used herein, a “precursor” anti-infective peptide and analogue or derivative thereof also includes a peptide having a complete primary amino acid sequence that is later synthetically modified using the methods described herein to generate a peptide having a structure similar or identical to a naturally produced peptide that has been post-translationally modified. In a preferred embodiment, any amino acid of a precursor anti-infective cationic peptide or anti-infective cationic peptide is chemically modified. A preferred modification of an anti-infective precursor cationic peptide is a carboxy-terminal amidation.

Many naturally occurring anti-infective peptides have an amide at their carboxy-terminal (R-CONH₂). Carboxy-terminal amidated peptides often exhibit improved antimicrobial activity (see, e.g., Peptide Res. 1:81, 1988; Proc. Natl. Acad. Sci. USA 86:9159,1989). A few recombinant expression systems have been reported to provide a carboxy-terminal amidated peptide; however, the model recombinant expression systems used to produce the amide form of proteins, such as mammalian cells and the baculovirus expression vector system, do so in low yields. These yields would be too low to efficiently and economically generate the commercial quantities of peptide amide that would be required. Several methods of enzymatic amidation of recombinant proteins to give a peptide amide have been explored (see, e.g., Nature 298:686-688,1982; J. Biol. Chem. 261:1815, 1986; Protein Eng. 1:195; U.S. Pat. No. 4,709,014). Disadvantageously, enzymatic amidation methods are costly and time consuming. That is, the process often yields unpredictable results, tends to be substrate specific, and requires multiple reaction steps to complete the modification, and may even require a further step of purifying the modified protein. Most yields with enzymatic amidation method were reported to be low (less than 25%). These shortcomings make the enzymatic transformation of a peptide carboxy-terminal acid to an amide an unacceptably expensive process for large-scale production of, for example, anti-infective peptides such as indolicidin analogue 11B7CN. In other embodiments, the amide group may be further modified with particularly desired R groups using methods known in the art.

An analogue or derivative may also be an anti-infective cationic peptide fusion protein. Fusion proteins, or chimeras, include fusions of one or more anti-infective cationic peptides, and fusions of cationic peptides with non-cationic peptides, such as anionic spacers and/or carriers. The peptides may also be labeled, such as with a radioactive label, a fluorescent label, a mass spectrometry tag, biotin, and the like.

The amino acid designations are herein set forth as either the standard one- or three-letter code. Unless otherwise indicated, a named amino acid refers to the L-enantiomer. Polar amino acids include asparagine (Asp or N) and glutamine (Gln or Q); as well as basic amino acids such as arginine (Arg or R), lysine (Lys or K), histidine (His or H), and derivatives thereof; and acidic amino acids such as aspartic acid (Asp or D) and glutamic acid (Glu or E), and derivatives thereof. Hydrophobic amino acids include tryptophan (Trp or W), phenylalanine (Phe or F), isoleucine (Ile or I), leucine (Leu or L), methionine (Met or M), valine (Val or V), and derivatives thereof; as well as other non-polar amino acids such as glycine (Gly or G), alanine (Ala or A), proline (Pro or P), and derivatives thereof. Amino acids of intermediate polarity include serine (Ser or S), threonine (Thr or T), tyrosine (Tyr or Y), cysteine (Cys or C), and derivatives thereof. A capital letter indicates an L-enantiomer amino acid; a small letter indicates a D-enantiomer amino acid. An anti-infective cationic peptide analogue or derivative thereof produced by the methods of the instant invention may include any one or combination of the above-noted alterations to a natural peptide, or any other modification known in the art.

Nucleic acid molecules encoding cationic peptides may be isolated from natural sources, may be obtained by automated synthesis of nucleic acid molecules, or may be obtained by using the polymerase chain reaction (PCR) with oligonucleotide primers having nucleotide sequences that are based upon known nucleotide sequences of anti-infective cationic peptide genes. In the latter approach, a cationic peptide gene is synthesized using mutually priming oligonucleotides (see, for example, Ausubel et al. (eds.), Short Protocols in Molecular Biology, 3^{rd} Edition, pages 8-8 to 8-9, John Wiley & Sons, 1995, herein after referred to as “Ausubel (1995)”). Established techniques using the polymerase chain reaction provide the ability to synthesize DNA molecules of at least two kilobases in length (Adang et al., Plant Molec. Biol. 21:1131, 1993; Bambot et al., PCR Methods and Applications 2:266, 1993; Dillon et al., “Use of the Polymerase Chain Reaction for the Rapid Construction of Synthetic Genes,” in Methods in Molecular Biology, Vol. 15: PCR Protocols: Current Methods and Applications, White (ed.), pages 263-268, Humana Press, Inc., 1993; Holowachuk et al., PCR Methods Appl. 4:299,1995). In addition, it is known in the art that nucleic acid molecules may be modified, without altering the amino acid sequence of an encoded protein or peptide, to optimize codons for translation in the particular host containing a nucleic acid molecule of interest.

Peptides may be synthesized by recombinant techniques (see e.g., U.S. Pat. No. 5,593,866) and a variety of host systems are suitable for production of the anti-infective peptides and analogues or derivatives thereof, including bacteria (e.g., E. coli), yeast (e.g., Saccharomyces cerevisiae), insect (e.g., Sf9), and mammalian cells (e.g., CHO, COS-7). Many expression vectors have been developed and are available for each of these hosts. In a preferred embodiment, vectors that are functional (i.e., capable of replicating) in bacteria are used in this invention. However, at times, it may be preferable to have vectors that are functional in other hosts or more than one host. Vectors and procedures for cloning and expression in E. coli are discussed herein and, for example, in Sambrook et al. (Molecular Cloning: A Laboratory Manual, Cold Spring Harbor Laboratory Press, Cold Spring Harbor, N.Y., 1987) and in Ausubel et al. (1995).

A DNA sequence encoding an anti-infective peptide may be introduced into an expression vector appropriate for a particular host. In preferred embodiments, the gene is cloned into a vector or expression vector to generate a fusion protein. The fusion partner may be chosen to contain an anionic region, such that a bacterial host is protected from the toxic effect of the peptide. This protective region may be a carrier or a spacer peptide that effectively neutralizes the antimicrobial effects of an anti-infective cationic peptide, and may also prevent peptide degradation by host proteases. The fusion partner carrier or spacer peptide of the invention may further function to transport the fusion peptide to inclusion bodies, the periplasm, the outer membrane, or the extracellular environment.

A fusion partner carrier suitable in the context of this invention specifically include, but are not limited to, cellulose binding domain (CBD), glutathione-S-transferase (GST), protein A from Staphylococcus aureus, two synthetic IgG-binding domains (ZZ) of protein A, outer membrane protein F, β-galactosidase (lacZ), and various products of bacteriophage λ and bacteriophage T7. From the teachings provided herein, it is apparent that other proteins may be used as carriers. Furthermore, the entire carrier protein need not be used. For example, in a preferred embodiment a fragment of CBD containing only 96 amino acids, which fragment is no longer capable of binding cellulose, is used as a carrier. A carrier fusion partner is optional unless it is also functioning as an anionic spacer peptide for an anti-infective cationic peptide, and again fragments of the carrier may be used as long as the protective anionic region is present.

Illustrative anionic spacer peptides may have the amino acid sequence of HEAEPEAEPIM, where the methionine residue can be used as a cleavage site. Similar naturally occurring examples of anionic spacer peptides include EAEPEAEP, EAKPEAEP, EAEPKAEP, EAESEAEP, EAELEAEP, EPEAEP and EAEP (Casteels-Josson, et al. EMBO J., 12:1569-1578, 1993). In preferred embodiments, the anionic spacer peptide is MEAEPEAEPIMEKR or MEAEPEAEPIMVKR, which provide a kexin cleavage site. In more preferred embodiments, the anionic spacer peptide is AEAEPEAEAEGK, AEAEPEAEAAGK, AEAEPEAEAEGPK, AEAEPEAEAAGPK, AEAEPELAEAAGK, AEAEPELVEAAGK, MAEAEPEAEPIMK, MAEAEPEAEEPIMK, MAEAEPEAEAPIMK, MAEAEPEAEPIMEK, or MAEAEPEAEPIMVK, which are spacers designed for cleavage with lysyl endopeptidase. Additional anionic spacer peptides are suitable for use in producing cationic peptides, such as doubles or other combinations of those illustrated above. When designing an anionic spacer peptide for expression of a particular cationic peptide as a fusion protein of the instant invention, the following criteria should be borne in mind: the negative charge of the anionic spacer peptide should substantially reduce the positive charge of the cationic peptide in the multi-domain fusion proteins; a cleavage point must be present at which the fusion protein will be specifically cleaved to give monomers of the desired cationic peptide; and the first 1-5 amino acids upstream of the lysyl peptidase cleavage site are preferably non-polar or hydrophobic amino acids.

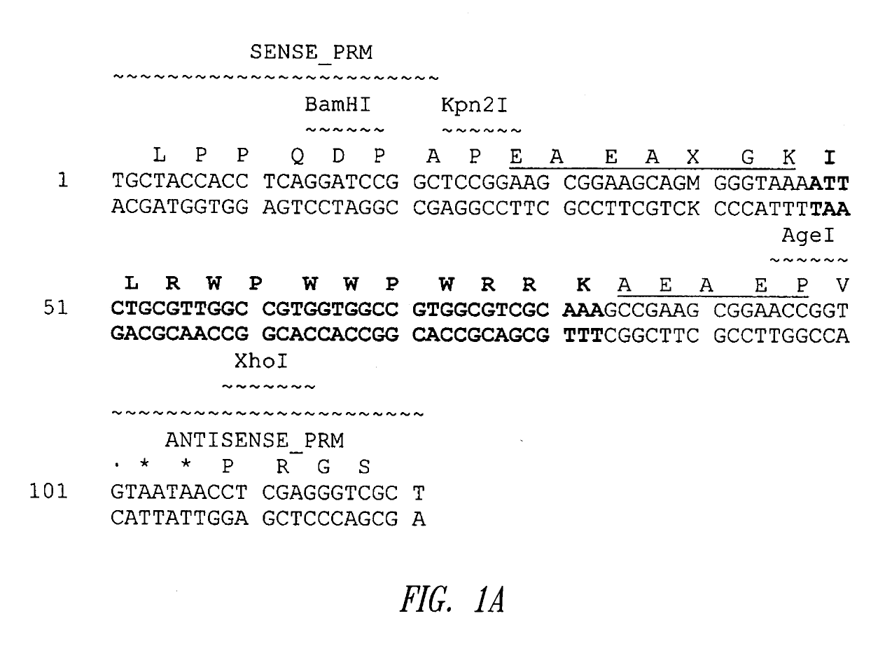

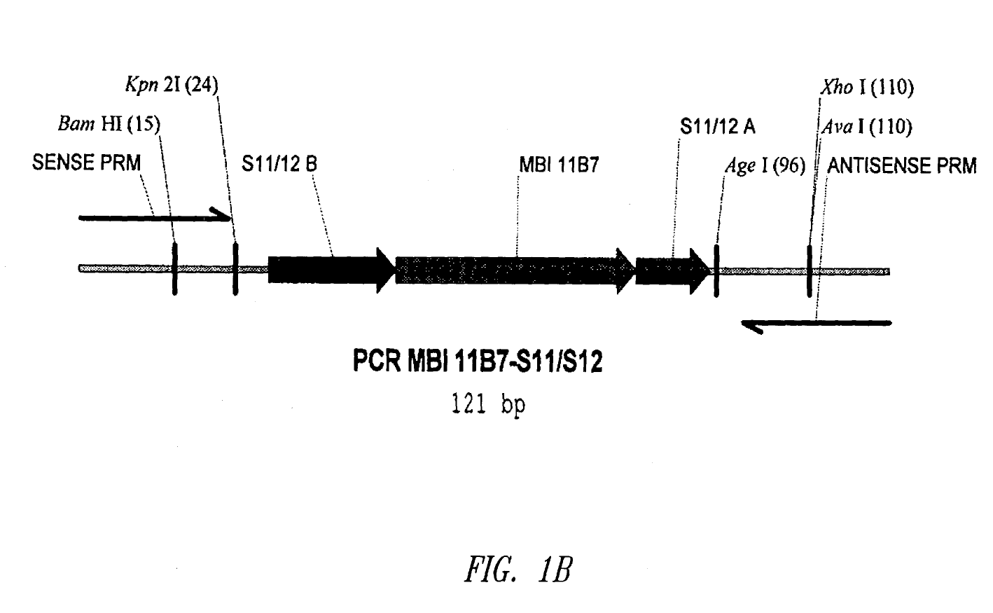

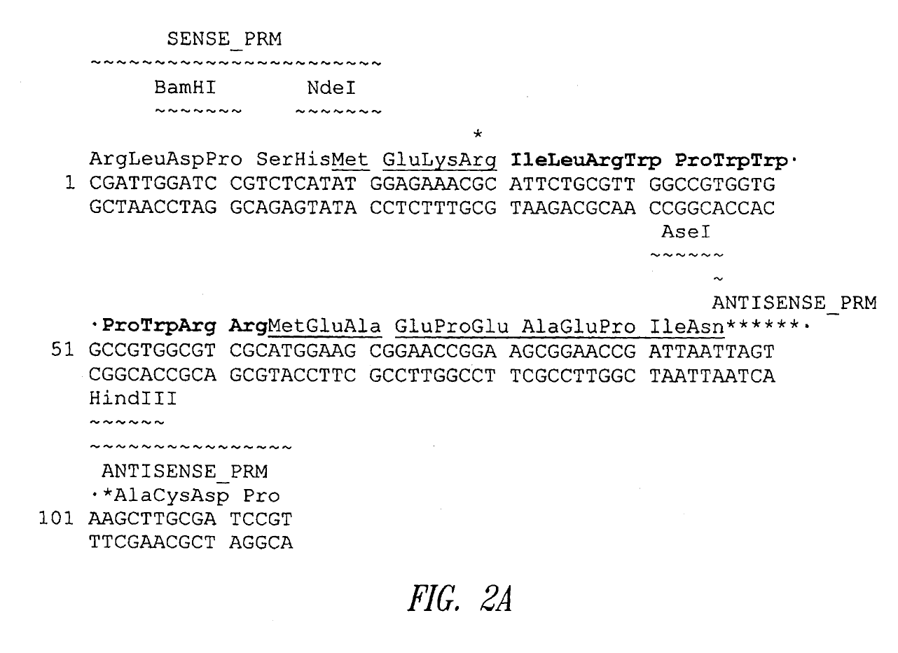

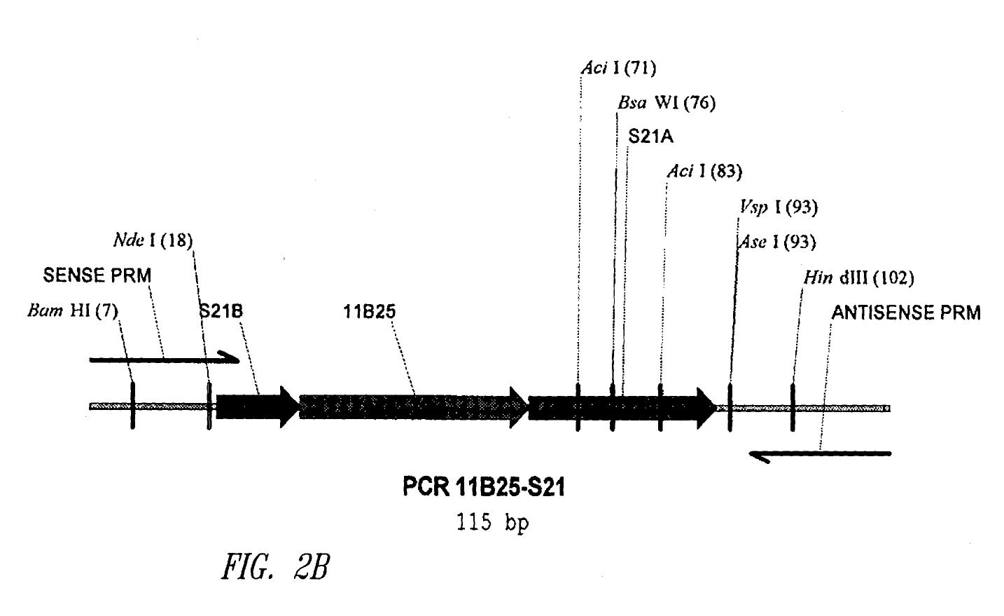

The anionic spacer peptide may be smaller, the same size, or larger than the cationic peptide. Preferably, such fusion proteins are designed with alternating units of anti-infective cationic peptide and anionic spacer peptide, although variations of such a configuration are also possible. In addition, a carrier as described herein may be optionally included as part of any fusion protein. In certain embodiments, a precursor anti-infective peptide or anti-infective peptide is synthetic or recombinant; preferably a precursor anti-infective peptide is recombinant. In a preferred embodiment, a recombinantly produced fusion protein comprises a nucleic acid molecule that encodes at least one precursor anti-infective peptide and at least one anionic spacer peptide (see, e.g., FIGS. 1 and 2). In other preferred embodiments, the units of precursor anti-infective cationic peptide (AICP) and anionic spacer peptide (ASP) are produced as a “template” having a structure, for example, of [AICP-ASP] or of [ASP-AICP-ASP], wherein the dashes represent a cleavage site. These templates can be combined in a variety of ways to produce multiple copies of anti-infective cationic peptides. Exemplary “templates” are shown in Table 1 (see, also Example 1).

TABLE 1

Exemplary Peptide with Spacer Templates

| | | | |
|---|---|---|---|
| | | | SEQ |
| | | | ID |
| Template | Oligo/Fragment | Nucleic Acid Sequence | NO. |

| | | |
|---|---|---|
| 1 | 11B7-S11/S12 | TGCTACCACC TCAGGATCCG GCTCCGGAAG |
| | | CGGAAGCAGM GGGTAAAATT CTGCGTTGGC |
| | | CGTGGTGGCC GTGGCGTCGC AAAGCCGAAG |
| | | CGGAACCGGT GTAATAACCT CGAGGGTCGC T |
| 2 | 11B7-S13/S14 | TGCTTAGGAT CCGAGCGGTC CGAAAATTCT |
| | | GCGTTGGCCG TGGTGGCCGT GGCGTCGCAA |
| | | AGCCGAAGCG GAACCGGAAG CGGAAGCAGM |
| | | GGGTCCTTAA TAAGCTTGGT ACCCCGATGC |
| | | TTG |
| 3 | Pvull frag. of pBS- | GCGGA AGCAGCGGGT AAAATTCTGC |
| | 11B7-S12 (for | GTTGGCCGTG GTGGCCGTGG CGTCGCAAAG |
| | S19/S20 | CCGAAGCGGA ACCGGTGTAA TAACCTCGAG |
| | amplification) | GGGGGGCCCG GTACCCAGCT TTTGTTCCCT |
| | | TTAGTGAGGG TTAATTGCGC GCTTGGCGTA |
| | | ATCATGGTCA TAGCTGTTTCC |
| 4 | 11B7-S10 | CGCCAGGGTT TTCCCAGTCA CGACGGATCC |
| | (for S29 and S32/33 | GTCTCATATG ATTCTGCGTT GGCCGTGGTG |
| | amplification) | GCCGTGGCGT CGCAAAATGG CCGAAGCGGA |
| | | ACCGGAAGCG GAACCGATTA ATTAAGCTTC |
| | | GATCCTCTAC GCCGGACGC |
| 5 | Ndel/Asel frag. of | TATGATTCTG CGTTGGCCGT GGTGGCCGTG |
| | pBCKS-V-11B7-S10 | GCGTCGCAAA ATGGCCGAAG CGGAACCGGA |
| | (for S30/31 and | AGCGGAACCG AT |
| | S34/35 amplification) | |

For expression in bacteria, such as E. coi, preferably the fusion protein is expressed in the form of an inclusion body. In such a case, the inclusion body must often be solubilized so that the precursor anti-infective peptide can be separated from the anionic spacer peptide by, for example, enzymatic cleavage with lysyl endopeptidase. Typically, inclusion bodies are solubilized with solubilization reagents well known in the art, including guanidine hydrochloride, SDS, and urea. However, these solubilization reagents were not effective for the present invention. Surprisingly, an alternate solubilization reagent, ammonium bicarbonate (NH₄HCO₃) was found to efficiently solubilize the fusion proteins of the invention. In a preferred embodiment, the recombinantly produced fusion protein is in an inclusion body and is solubilized with about 50 mM to about 500 mM NH₄HCO₃, preferably about 75 mM to about 250 mM NH₄HCO₃, and most preferably about 100 mM to about 125 mM NH₄HCO₃. In addition, the NH₄HCO₃ solution is at a pH ranging from about pH 7 to pH 9.5, and most preferably at about a pH 8.5 to about pH 9.0. The use of solubilization reagent NH₄HCO₃ is especially useful when the fusion protein is to be cleaved with lysyl endopeptidase. Following cleavage to release the final product (i.e. precursor anti-infective peptide), there is no requirement for renaturation of the peptide.

In the present invention, the DNA cassette to be expressed, comprising a fusion protein sequence and cationic peptide nucleic acid sequence, may be inserted into an expression vector, which vector can be a plasmid, virus, or other vehicle known in the art. Preferably, the expression vector is a plasmid that contains an inducible or constitutive promoter (i.e., an expression control element) to facilitate the efficient transcription of the inserted DNA sequence in the host. Transformation of the host cell with the recombinant DNA may be carried out by Ca⁺⁺-mediated techniques, by electroporation, or other methods well known to those skilled in the art. At minimum, an expression vector should contain a promoter sequence. However, other regulatory sequences may also be included. Such sequences include an enhancer, ribosome binding site, transcription termination signal sequence, secretion signal sequence, origin of replication, selectable marker, and the like. The regulatory sequences are operably linked with one another to allow transcription and subsequent translation.

In preferred aspects, the plasmids used herein for expression include an expression control element designed for expression of the proteins in bacteria. Suitable promoters, including both constitutive and inducible promoters, are widely available and are well known in the art. Commonly used promoters for expression in bacteria include promoters from T7, T3, T5, and SP6 phages, and the trp, lpp, and lac operons. Hybrid promoters (see, U.S. Pat. No. 4,551,433), such as tac and trc, may also be used. Examples of plasmids for expression in bacteria include any of the pET series of expression vectors, such as pET 24a(+) (see, e.g., U.S. Pat. No. 4,952,496; available from Novagen, Madison, Wis.). Low copy number vectors (e.g., pPD100) can be used for efficient overproduction of peptides deleterious to the E. coli host (Dersch et al., FEMS Microbiol. Lett. 123: 19, 1994). Bacterial hosts for the T7 expression vectors may contain chromosomal copies of DNA encoding T7 RNA polymerase operably linked to an inducible promoter (e.g., lacUV promoter; see, U.S. Pat. No. 4,952,496), such as found in the E. coli strains HMS174(DE3)pLysS, BL21(DE3)pLysS, HMS174(DE3) and BL21(DE3). T7 RNA polymerase can also be present on plasmids compatible with the T7 expression vector. The polymerase may be under control of a lambda promoter and repressor (e.g., pGP1-2; Tabor and Richardson, Proc. Natl. Acad. Sci. USA 82: 1074, 1985).

To facilitate isolation of the cationic peptide sequence, amino acids susceptible to chemical cleavage (e.g., CNBr) or enzymatic cleavage (e.g., V8 protease, trypsin, lysyl endopeptidase) should be used to bridge the peptide and fusion partner. The determination and design of the amino acid sequence of the cleavage site is highly dependent on the strategy of cleavage and the amino acid sequence of the cationic peptide, anionic spacer peptide and carrier protein. The removal of the cationic peptide can be accomplished through any known chemical or enzymatic cleavages specific for peptide bonds. Chemical cleavages include (R. A. Jue & R. F. Doolittle, Biochemistry, (1985) 24: 162-170; R. L. Lundblad, Chemical Reagents for Protein Modification (CRC Press, Boca Raton, Fla.; 1991), Chapter 5.), but are not limited to those treated by cyanogen bromide cleavages at methionine (Met↓), N-chlorosuccinimide or o-iodosobenzoic acid at tryptophan (Trp↓), hydroxylamine at asparaginyl-glycine bonds (Asn↓Gly), or low pH at aspartyl-proline bonds (Asp↓Pro). Alternatively, there are a vast number of proteases described in the literature, but the majority has little specificity for a cleavage site. Enzymatic cleavages that can be performed include without limitation those catalyzed by Factor Xa, Factor XIIa, kexin, thrombin, enterokinase, collagenase, Staphylococcus aureus V8 protease (endoproteinase Glu-C), endoproteinase Arg-C, lysyl endopeptidase (endoproteinase Lys-C), chymotrypsin, and trypsin.

The precursor anti-infective cationic peptides or analogues and derivatives thereof of the instant invention may be any of the cationic peptides provided herein or known or yet to be known in the art that are recombinantly produced with one or more amino acids deleted, added, or modified. Accordingly, single amino acids, peptides, or polypeptides may be synthesized by standard chemical methods, including synthesis by automated procedure to have a particular modification as described above. In addition, the single amino acid, peptides, or polypeptides may include less common, non-natural amino acids (such as D-amino acids). In general, modified or uncommon amino acid and peptide analogues to be coupled to the recombinant precursor anti-infective cationic peptides are synthesized based on the standard solid-phase Fmoc protection strategy with HATU as the coupling agent. The peptide is cleaved from the solid-phase resin with trifluoroacetic acid containing appropriate scavengers, which also deprotects side chain functional groups. Crude modified or unusual amino acid and peptide products may be further purified using preparative reversed-phase chromatography. Other purification methods, such as partition chromatography, gel filtration, gel electrophoresis, or ion-exchange chromatography may be used. Other synthesis techniques, known in the art, such as the tBoc protection strategy, or use of different coupling reagents and the like can be employed to produce equivalent peptides. In addition, the molecular mass or sequence of modified precursor anti-infective cationic peptides may be verified by a variety of standard and high throughput techniques known in the art. For example, verification of a modified peptide may be determined by peptide mass mapping by matrix assisted laser desorption ionization time of flight (MALDI-TOF) mass spectrometry, and peptide sequence can be determined by post source decay (PSD) MADLI-MS or liquid chromatography tandem mass spectrometry (LC-MS/MS).

The present invention also provides methods for treating and preventing infections by administering to a patient a therapeutically effective amount of an anti-infective peptide, preferably an indolicidin or analogue or derivative thereof, as described herein. The peptide is preferably part of a pharmaceutical composition when used in the methods of the present invention. The pharmaceutical composition will include at least one of a pharmaceutically acceptable vehicle, carrier, diluent, or excipient, in addition to one or more anti-infective peptide and, optionally, other components. Pharmaceutically acceptable excipients for therapeutic use are well known in the pharmaceutical art, and are described herein and described, for example, in Remington's Pharmaceutical Sciences, Mack Publishing Co. (A. R. Gennaro, ed., 18^{th} Edition, 1990) and in CRC Handbook of Food, Drug, and Cosmetic Excipients, CRC Press LLC (S. C. Smolinski, ed., 1992).

The therapeutic efficacy of a peptide composition according to the present invention is based on a successful clinical outcome and does not require 100% elimination of the microorganisms involved in the infection. Achieving a level of anti-infective activity at the site of infection that allows the host to survive, resolve the infection, or eradicate the causative agent is sufficient. When host defenses are maximally effective, such as in an otherwise healthy individual, only a minimal anti-infective effect may suffice. Thus, for anti-microbial activity, reducing the organism load by even one log (a factor of 10) may permit the defenses of the host to control the infection. In addition, clinical therapeutic success may depend more on augmenting an early bactericidal effect rather than on a long-term effect because this allows time for activation of host defense mechanisms. This is especially true for life-threatening infections (e.g., meningitis) and other serious chronic infections (e.g., infective endocarditis). Similarly, the anti-inflammatory activity could aid in keeping excessive host defense mechanism reactions from causing additional damage.

The formulations of the present invention, having an amount of an anti-infective peptide sufficient to treat, prevent, or ameliorate an infection or inflammation are, for example, particularly suitable for topical (e.g., creams, ointments, skin patches, eye drops, ear drops, shampoos) application or administration. Other typical routes of administration include, without limitation, oral, parenteral, sublingual, bladder wash-out, vaginal, rectal, enteric, suppository, nasal, and inhalation. The term parenteral, as used herein, includes subcutaneous, intravenous, intramuscular, intraarterial, intraabdominal, intraperitoneal, intraarticular, intraocular or retrobulbar, intraaural, intrathecal, intracavitary, intracelial, intraspinal, intrapulmonary or transpulmonary, intrasynovial, and intraurethral injection or infusion techniques. The pharmaceutical compositions of the present invention are formulated to allow the anti-infective peptide contained therein to be bioavailable upon administration of the composition to a subject. The level of peptide in serum and other tissues after administration can be monitored by various well-established techniques, such as bacterial, chromatographic or antibody based (e.g., ELISA) assays. Thus, in certain preferred embodiments, anti-infective peptides and analogues and derivatives thereof, as described herein, are formulated for topical application to a target site on a subject in need thereof, such as an animal or a human.

The compositions may be administered to a subject as a single dosage unit (e.g., a tablet, capsule, or gel), and the compositions may be administered as a plurality of dosage units (e.g., in aerosol form). For example, the anti-infective peptide formulations may be sterilized and packaged in single-use, plastic laminated pouches or plastic tubes of dimensions selected to provide for routine, measured dispensing. In one example, the container may have dimensions anticipated to dispense 0.5 ml of the an anti-infective peptide composition (e.g., a gel form) to a limited area of the target surface on or in a subject to treat or prevent an infection. A typical target, for example, is in the immediate vicinity of the insertion site of an intravenous catheter or intraarticularly at the joint that has arthritis.

An anti-infective peptide composition may be provided in various forms, depending on the amount and number of different pharmaceutically acceptable excipients present. For example, the peptide composition may be in the form of a solid, a semi-solid, a liquid, a lotion, a cream, an ointment, a cement, a paste, a gel, or an aerosol. In a preferred embodiment, the peptide formulation is in the form of a gel. The pharmaceutically acceptable excipients suitable for use in the peptide formulation compositions as described herein may include, for example, a viscosity-increasing agent, a buffering agent, a solvent, a humectant, a preservative, a chelating agent, an oleaginous compound, an emollient, an antioxidant, an adjuvant, and the like. The function of each of these excipients is not mutually exclusive within the context of the present invention. For example, glycerin may be used as a solvent or as a humectant or as a viscosity-increasing agent. In one preferred embodiment, the formulation is a composition comprising an anti-infective peptide, a viscosity-increasing agent, and a solvent, which is useful, for example, at a target site having inflammation and/or an infection associated with an implanted or indwelling medical device, as described herein.

Solvents useful in the present compositions are well known in the art and include without limitation water, glycerin, propylene glycol, isopropanol, ethanol, and methanol. In some embodiments, the solvent is glycerin or propylene glycol, preferably at a concentration ranging from about 0.1% to about 20%, more preferably about 5% to about 15%, and most preferably about 9% to 11%. In other embodiments, the solvent is water or ethanol, preferably at a concentration up to about 99%, more preferably up to about 90%, and most preferably up to about 85%. (Unless otherwise indicated, all percentages are on a w/w basis.) In yet other embodiments, the solvent is at least one of water, glycerin, propylene glycol, isopropanol, ethanol, and methanol, preferably is glycerin or propylene glycol and ethanol, more preferably is glycerin and ethanol, and most preferably is glycerin and water. One embodiment is a composition comprising a anti-infective peptide, a viscosity-increasing agent, a solvent, wherein the solvent comprises at least one of water at a concentration up to 99%, glycerin at a concentration up to 20%, propylene glycol at a concentration up to 20%, ethanol at a concentration up to 99%, and methanol at a concentration up to 99%.

Another useful pharmaceutical excipient of the present invention is a viscosity-increasing agent. In certain embodiments, the anti-infective peptide compositions of the present invention include a viscosity-increasing agent, including without limitation dextran, polyvinylpyrrolidone, methylcellulose, carboxymethyl cellulose, hydroxyethyl cellulose, hydroxypropyl methylcellulose, and hydroxypropyl cellulose, and combinations thereof. In preferred embodiments, the viscosity-increasing agent is hydroxyethyl cellulose or hydroxypropyl methylcellulose, preferably at a concentration ranging from about 0.5% to about 5%, more preferably from about 1% to about 3%, most preferably from about 1.3% to about. 1.7%. In yet other preferred embodiments, the peptide compositions have a first viscosity-increasing agent, such as hydroxyethyl cellulose, hydroxypropyl methylcellulose, dextran, or polyvinylpyrrolidone, and a second viscosity-increasing agent such as hydroxyethyl cellulose, hydroxypropyl methylcellulose, dextran, or polyvinylpyrrolidone. When used as either a first or second viscosity-increasing agent, dextran and polyvinylpyrrolidone are preferably used at a concentration ranging from about 0.1% to about 5% and more preferably from about 0.5% to about 1%. In one preferred embodiment, the first viscosity-increasing agent is hydroxyethyl cellulose at a concentration up to 3% and the second viscosity-increasing agent is hydroxypropyl methylcellulose at a concentration up to 3%. As is known in the art, the amount of viscosity-increasing agent may be increased to shift the form of the composition from a liquid to a gel to a semi-solid form. Thus, the amount of a-viscosity-increasing agent used in a formulation may be varied depending on the intended use and location of administration of the peptide compositions provided herein.

In certain applications, it may be desirable to maintain the pH of the peptide composition contemplated by the present invention within a physiologically acceptable range and within a range that optimizes the activity of the peptide or analogue or derivative thereof. For example, the cationic peptides of the present invention function best in a composition that is neutral or somewhat acidic, although the peptides will still have antimicrobial and anti-inflammatory activity in a composition that is slightly basic (i.e., pH 8). Accordingly, a composition comprising a peptide, a viscosity-increasing agent, and a solvent, may further comprise a buffering agent. In certain embodiments, the buffering agent comprises a monocarboxylate or a dicarboxylate, and more specifically may be acetate, fumarate, lactate, malonate, succinate, or tartrate. Preferably, the peptide composition including the buffering agent has a pH ranging from about 3 to about 8, and more preferably from about 3.5 to about 7. In another preferred embodiment, the buffering agent is at a concentration ranging from about 1 mM to about 200 mM, and more preferably from about 2 mM to about 20 mM. and most preferably about 4 mM to about 6 mM.

Other optional pharmaceutically acceptable excipients are those that may, for example, aid in the administration of the formulation (e.g., anti-irritant, polymer carrier, adjuvant) or aid in protecting the integrity of the components of the formulation (e.g., anti-oxidants and preservatives). Additionally, for example, a 1.0% cationic peptide composition may be stored at 2° C. to 8° C. In certain embodiments, the composition comprising a peptide, a viscosity-increasing agent, and a solvent, may further comprise a humectant (preferably sorbitol, glycerol, and the like) or a preservative (preferably benzoic acid, benzyl alcohol, phenoxyethanol, methylparaben, propylparaben, and the like). As used herein, any reference to an acid may include a free acid, a salt, and any ester thereof. In other embodiments, any of the aforementioned compositions further comprise a humectant and a preservative. In certain circumstances, the peptide or analogue or derivative thereof may itself function as a preservative of the final therapeutic composition. For example, a preservative is optional in the gel formulations described herein because the gels may be sterilized by autoclaving and, furthermore, show the surprising quality of releasing (i.e., making bioavailable) a peptide at a more optimal rate than other formulations, such as a cream. In addition, particular embodiments may have in a single formulation a humectant, a preservative, and a buffering agent, or combinations thereof. Therefore, a preferred embodiment is a composition comprising a peptide, a viscosity-increasing agent, a solvent, a humectant, and a buffering agent. Another preferred embodiment is a composition comprising a peptide, a viscosity-increasing agent, a buffering agent, and a solvent. In yet another preferred embodiment, the composition comprises a peptide, a buffering agent, and a solvent. Each of the above formulations may be used to treat, prevent, or ameliorate infection, to reduce the microflora at a target site such as a catheter insertion site on a subject (i.e., animal or human), or to reduce inflammation at a target site.

In yet other embodiments, the composition is in the form of an ointment comprising an anti-infective peptide (preferably in an amount sufficient to treat or prevent an infection) and an oleaginous compound. For example, oleaginous compound may be petrolatum. In one embodiment, the oleaginous compound is present at a concentration ranging from about 50% to about 100%, more preferably from about 70% to about 100%, even more preferably from about 80% to about 100%, and most preferably from about 95% to about 100%. In certain other embodiments, the ointment composition may further comprise at least one emollient. The emollients may be present at a concentration ranging from about 1% to about 40%, more preferably from about 5% to about 30%, and more preferably from about 5% to about 10%. In certain preferred embodiments, the emollient may be mineral oil, cetostearyl alcohol, glyceryl stearate, and a combination thereof.

In another aspect the composition may be in the form of a semi-solid emulsion (e.g., a cream) comprising an anti-infective peptide (preferably in an amount sufficient to treat or prevent an infection), a solvent, a buffering agent, at least one emollient, and at least one emulsifier. In a preferred embodiment, the semi-solid emulsion or cream further comprises at least one of a humectant (e.g., sorbitol and/or glycerin), an oleaginous compound (e.g., petrolatum), a viscosity increasing agent (e.g., dextran, polyvinylpyrrolidone, hydroxyethyl cellulose, and/or hydroxypropyl methylcellulose), an anti-oxidant (e.g., butylated hydroxytoluene and preferably at a concentration ranging from about 0.01% to about 0.1%), a preservative (e.g., benzoic acid, benzyl alcohol, phenoxyethanol, methylparaben, propylparaben, or a combination thereof), or a combination thereof. In certain preferred embodiments, the emollient may be one or more of stearyl alcohol, cetyl alcohol, and mineral oil. In certain other preferred embodiments, the emulsifiers may be one or more of stearyl alcohol, cetyl alcohol, polyoxyethylene 40 stearate, and glyceryl monostearate. In a preferred embodiment, the emulsifier is present at a concentration ranging from about 1% to about 20%, more preferably from about 5% to about 10, and most preferably from about 1% to about 1.5%. As noted above, the function of each of these emulsifiers and emollients is not mutually exclusive in that an emollient may function as an emulsifier and the emulsifier may function as an emollient, depending on the particular formulation, as is known in the art and is described herein. In certain preferred embodiments the solvent comprises water and the like, and the buffering agent comprises a monocarboxylate or dicarboxylate and the like, as described herein.

A subject suitable for treatment with a peptide formulation may be identified by well-established indicators of risk for developing a disease or well-established hallmarks of an existing disease. For example, indicators of an infection include fever, pus, microorganism positive cultures, inflammation, and the like. Infections that may be treated with peptides provided by the present invention include without limitation those caused by or due to microorganisms, whether the infection is primary, secondary, opportunistic, or the like. Examples of microorganisms include bacteria (e.g., Gram-positive, Gram-negative), fungi, (e.g., yeast and molds), parasites (e.g., protozoans, nematodes, cestodes and trematodes), viruses (e.g., HIV, HSV, VSV), algae, and prions. Specific organisms in these classes are well known (see, for example, Davis et al., Microbiology, 3^{rd} edition, Harper & Row, 1980; and Stanier et al., The Microbial World, 5^{th} edition, Prentice Hall, 1986). Infections include, but are not limited to, toxic shock syndrome, diphtheria, cholera, typhus, meningitis, whooping cough, botulism, tetanus, pyogenic infections, sinusitis, pneumonia, gingivitis, mucitis, folliculitis, cellulitis, acne and acne vulgaris, impetigo, osteomyelitis, endocarditis, ulcers, burns, dysentery, urinary tract infections, gastroenteritis, anthrax, Lyme disease, syphilis, rubella, septicemia, and plague; as well as primary, secondary, and opportunistic infections associated with, for example, trauma, surgery, endotracheal intubation, tracheostomy, and cystic fibrosis.

A subject may have other clinical indications that have associated infection or inflammation treatable or preventable with the compositions and methods of the present invention, which include without limitation those associated with implantable, indwelling, or similar medical devices, such as intravascular catheters (e.g., intravenous and intra-arterial), right heart flow-directed catheters, Hickman catheters, arterioyenous fistulae, catheters used in hemodialysis and peritoneal dialysis (e.g., silastic, central venous, Tenckhoff, and teflon catheters), vascular access ports, indwelling urinary catheters, urinary catheters, silicone catheters, ventricular catheters, synthetic vascular prostheses (e.g., aortofemoral and femoropopliteal), prosthetic heart valves, prosthetic joints, orthopedic implants, penile implants, shunts (e.g., Scribner, Torkildsen, central nervous system, portasystemic, ventricular, ventriculoperitoneal), intrauterine devices, tampons, contact lenses, dental implants, ureteral stents, pacemakers, implantable defibrillators, tubing, cannulas, probes, blood monitoring devices, needles, and the like. As used herein, “medical device” refers to any device for use in a subject, such as an animal or human.

All of the above U.S. patents, U.S. patent application publications, U.S. patent applications, foreign patents, foreign patent applications, and non-patent publications referred to in this specification and/or listed in the Application Data Sheet, are incorporated herein by reference, in their entirety.

The following Examples are provided by way of illustration and not by way of limitation.

### EXAMPLES

### Example 1

### Production of Nucleic Acid Templates Encoding a Fusion Protein Containing a Precursor Cationic Peptide

A nucleic acid expression construct can be generated to produce precursor cationic peptides, which can then be modified (e.g., amidated). As discussed in more detail below, nucleic acid sequence “templates” encoding at least one precursor indolicidin analogue (e.g., 11B7, ILRWPWWPWRRK or 11B25, ILRWPWWPWRR) cationic peptide and at least one anionic spacer peptide were generated for use in a nucleic acid expression construct encoding a fusion protein that would not be toxic to the host containing the construct.

In one embodiment, a nucleic acid cassette encoding a fusion protein containing a single, full-length precursor indolicidin analogue was produced having a structure of [spacer-precursor cationic peptide-spacer]. In particular, specific nucleic acid primers (FOR 11B7-S11: 5′-TGCTACCACCTCAGGATCCGGCT-3′ and REV 11B7-S11: 5′-AGCGACCCTCGAGGTTATTACA-3′) were synthesized and used to amplify by polymerase chain reaction (PCR) nucleic acid Template 1 (see Table 1 and FIG. 1), which amplified template can be cloned into an expression vector to produce an 11B7 fusion protein. The full-length precursor indolicidin analogue is subsequently isolated and modified as described herein (e.g., amidated at the carboxy-terminus).

In another embodiment, a similar nucleic acid cassette encoding a fusion protein containing a single precursor indolicidin analogue was produced, but this template encodes a peptide lacking one carboxy-terminal amino acid. In particular, specific nucleic acid primers (FOR 11B25: 5′-CGATTGGATCCGTCTCA TATG-3′; REV 11B25: 5′-ACGGATCGCAAGCTTACTAA-3′) were synthesized and used to amplify by PCR the nucleic acid template encoding an 11B25 fusion protein (FIG. 2). The indolicidin analogue lacking one carboxy-terminal amino acid is subsequently isolated and modified by chemically adding a non-natural amino acid (e.g., an amidated amino acid), as described herein.

In other embodiments, nucleic acid primers (FOR 11B7-S13/S14: 5′-TG CTTAGGATCCGAGCGGTCCG-3′ and REV 11B7-S13/S14: 5′-CAAGCATCGGGGTA CCAAGC-3′) were used to amplify by PCR nucleic acid Template 2 (Table 1). In yet other embodiments, primers having mutations were used to create nucleic acid templates having a mutation. For example, primers having mutations are depicted with the mutation shown in brackets and the region of primer sequence that hybridizes to the nucleic acid template being underlined. Primer S19/20 FOR: 5′-CACTCTCCGGAACTG(GYG)GAAGCAGCGGGTAAAATT-3′ was used with M13 Reverse (5′-GGAAACAGCTATGACCATG-3′) to amplify by PCR Template 3 (Table 1). Primers S29F: 5′-ACTCACCATATG(AAA)ATTCTGCGTTGGC CGTGGT-3′ and CBD-180U: 5′-GCGTCCGGCGTAGAGGATCG-3′ were used to amplify by PCR Template 4 (Table 1). Primer S29F was then used with S30R: 5′-TTCAAAGCTTAAT TAATCGG(TKC)TTCCGCTTCCGGTTCCGC-3′ to amplify by PCR Template 5 (Table 1). Primer S32/33F:5′-ACTCACCATATG(GWGAAA)ATTCTGCGTTGGCCGTGGT-3′ was used with CBD-180U to amplify by PCR Template 4 (Table 1). Primer S34/35: 5′-ACTCACCATATG(AAG)ATTCTGCGTTGGCCGTGGT-3′ was used with S30R to amplify by PCR Template 5 (Table 1).

### Example 2

### Nucleic Acid Expression Construct Encoding a Fusion Protein Containing a Precursor Cationic Peptide

The templates generated as described in Example 1 were cloned into pBCKS-V, a pBluescript-based vector that lacks an AseI (or isoschizomer VspI) restriction site, to facilitate multimerization cloning using restriction enzymes such as BamHI and HindIII. Alternatively, other available cloning vectors such as pBluescript (Stratagene, La Jolla, Calif.) could be used. The resulting indolicidin analogue/spacer fusion protein-coding cassettes were confirmed by nucleic acid sequencing and comprise the following amino acid sequences, each with differing charge and hydrophobicity, as shown in Table 2.

TABLE 2

Exemplary Precursor Peptide-Anionic
Spacer Fusion Proteins

| | | |
|---|---|---|
| | Sequence (anti-infective | SEQ |
| Fusion Protein | peptide-anionic spacer) | ID NO. |

| | |
|---|---|
| 11B7-S11 | ILRWPWWPWRRK-AEAEPEAEAEGK |
| 11B7-S12 | ILRWPWWPWRRK-AEAEPEAEAAGK |
| 11B7-S13 | ILRWPWWPWRRK-AEAEPEAEAEGPK |
| 11B7-S14 | ILRWPWWPWRRK-AEAEPEAEAAGPK |
| 11B7-S19 | ILRWPWWPWRRK-AEAEPELAEAAGK |
| 11B7-S20 | ILRWPWWPWRRK-AEAEPELVEAAGK |
| 11B7-S29 | ILRWPWWPWRRK-MAEAEPEAEPIMK |
| 11B7-S30 & 34 | ILRWPWWPWRRK-MAEAEPEAEEPIMK |
| 11B7-S31 & 35 | ILRWPWWPWRRK-MAEAEPEAEAPIMK |
| 11B7-S32 | ILRWPWWPWRRK-MAEAEPEAEPIMEK |
| 11B7-S33 | ILRWPWWPWRRK-MAEAEPEAEPIMVK |
| 11B25-S21 | ILRWPWWPWRR-MEAEPEAEPIMEKR |
| 11B25-S22 | ILRWPWWPWRR-MEAEPEAEPIMVKR |

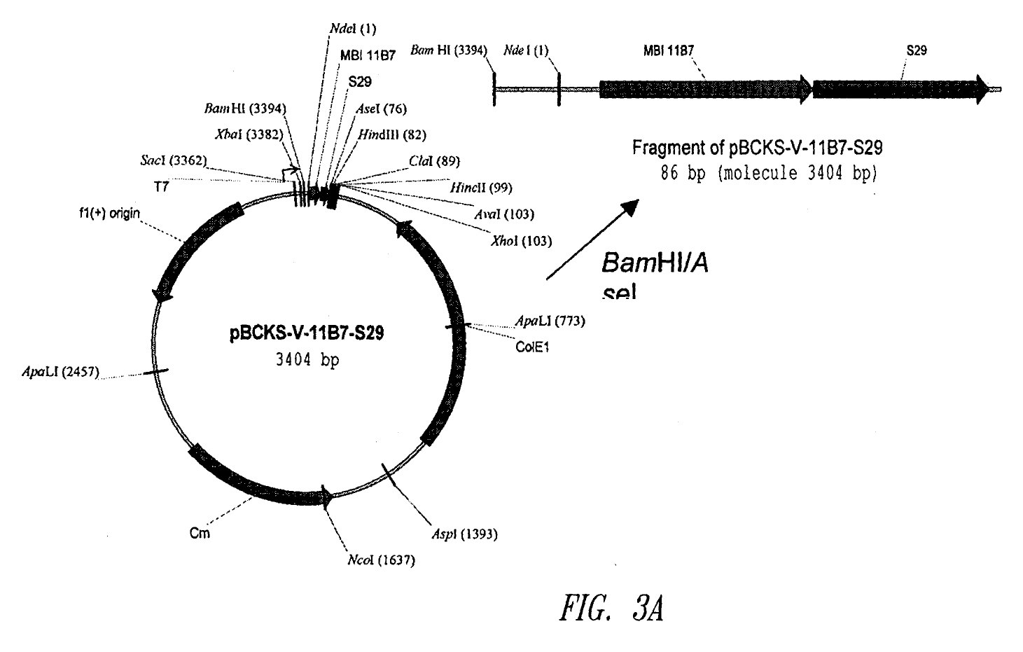

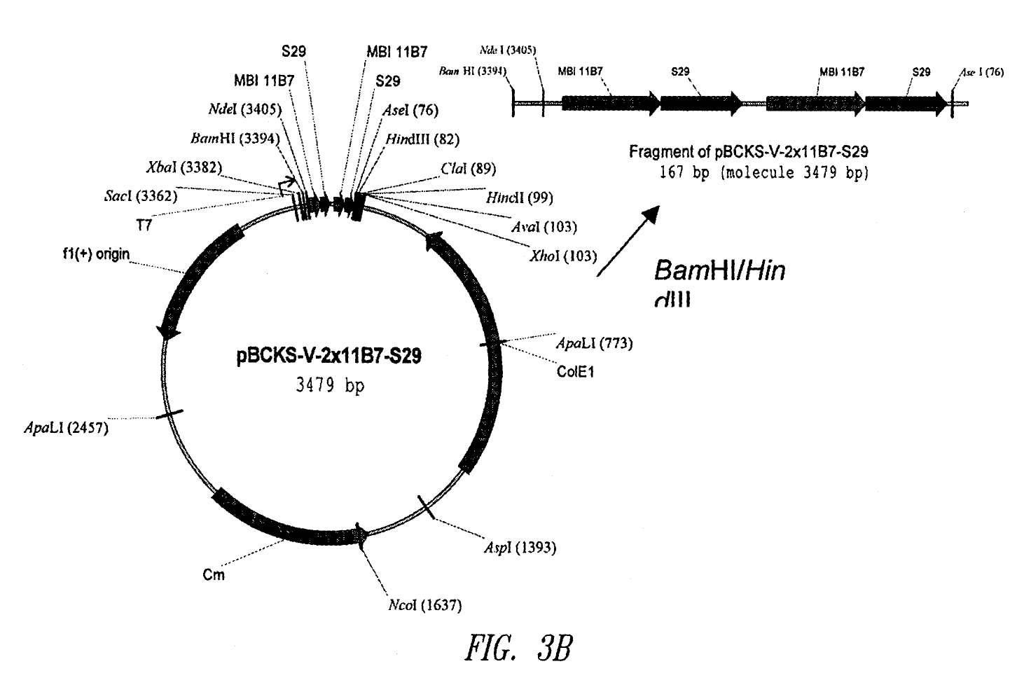

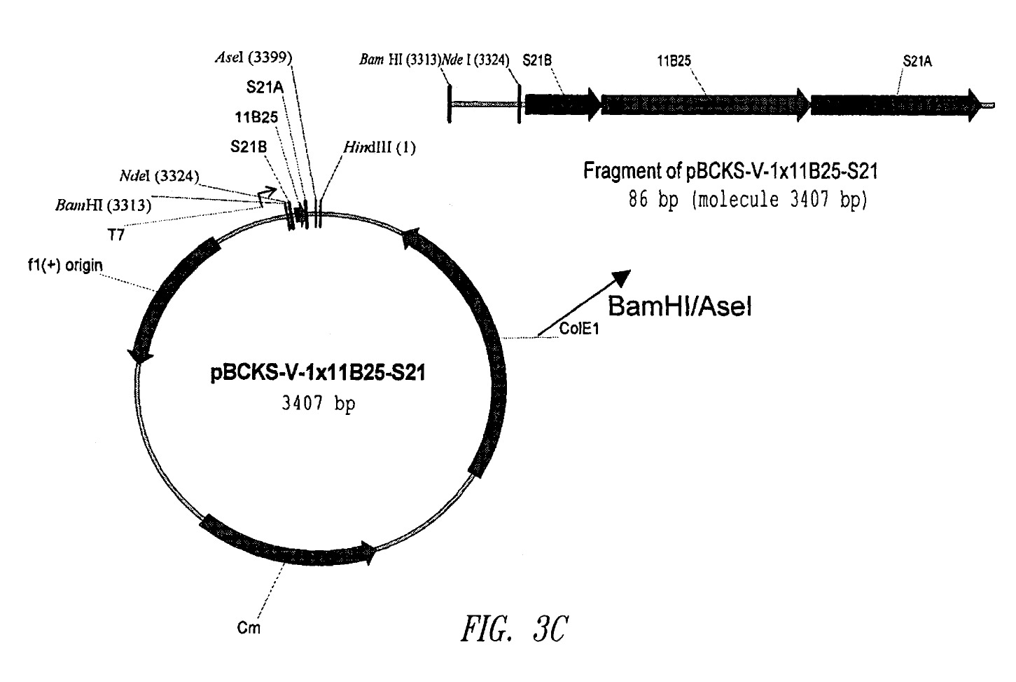

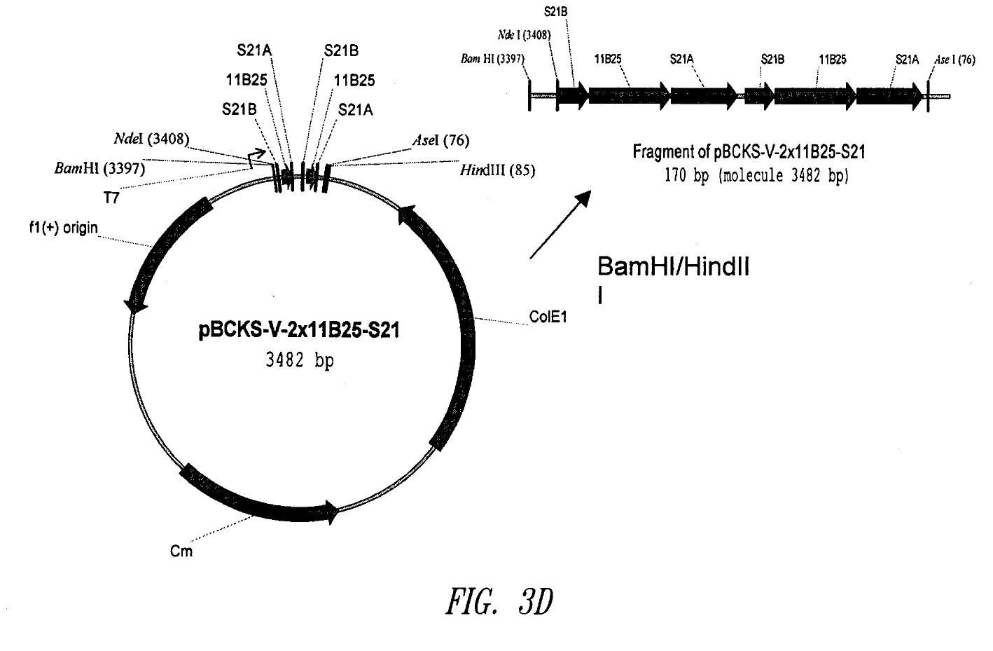

Multimerization cloning was then used to produce nucleic acid constructs having multiple copies of the precursor indolicidin analogue/anionic spacer peptide fusion protein-encoding cassette (FIG. 3). In this example, to generate a nucleic acid construct having two indolicidin analogue/spacer fusion protein cassettes (2x), pBCKS-V-1x11B7-S29 or pBCKS-V-1x11B25-S21 was digested with the restriction enzymes BamHI and AseI and the resultant cassette fragment was cloned into the vector fragment of pBCKS-V-1x11B7-S29 or pBCKS-V-1x11B25-S21, respectively, digested with the restriction enzymes BamHI and NdeI. The resultant construct, pBCKS-V-2x11B7-S29 or pBCKS-V-2x11B25-S21, was then used in the same cloning strategy to obtain a nucleic acid construct having four cassettes (4x). The 4x construct was then used in conjunction with the 1x construct to generate a 5x nucleic acid construct. The 5x construct was used to generate 10x and 15x nucleic acid constructs. Alternatively, the multimerization cloning enzymes Kpn2I and AgeI or RsrII and Eco019I can be used with enzymes such as BamHI and XhoI or HindIII to produce multimers of nucleic acid constructs.

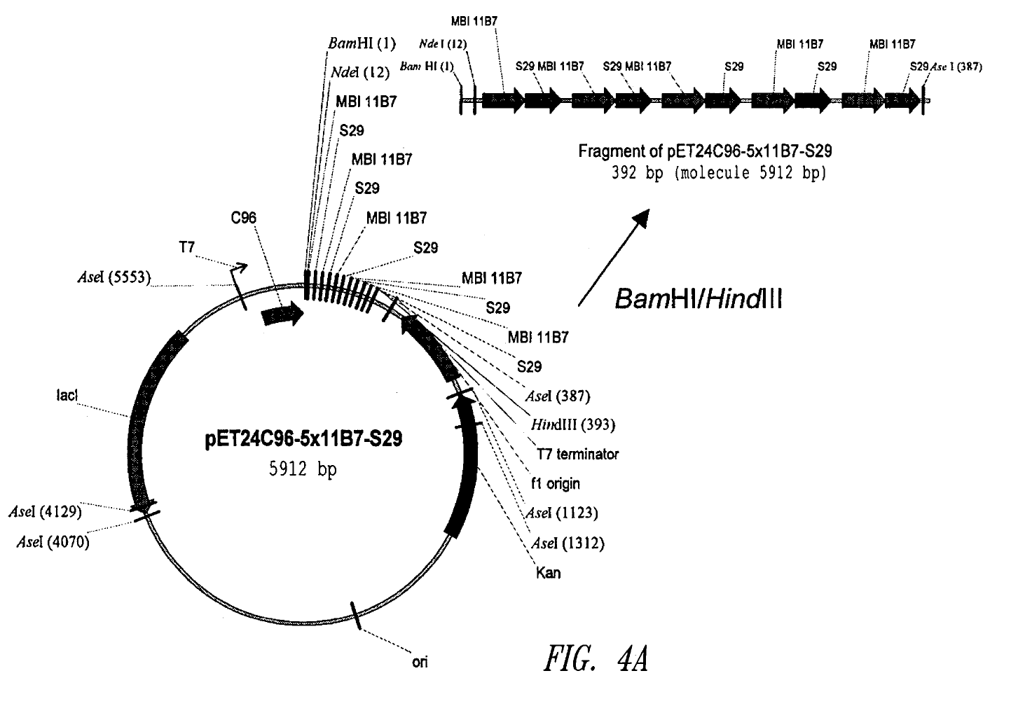

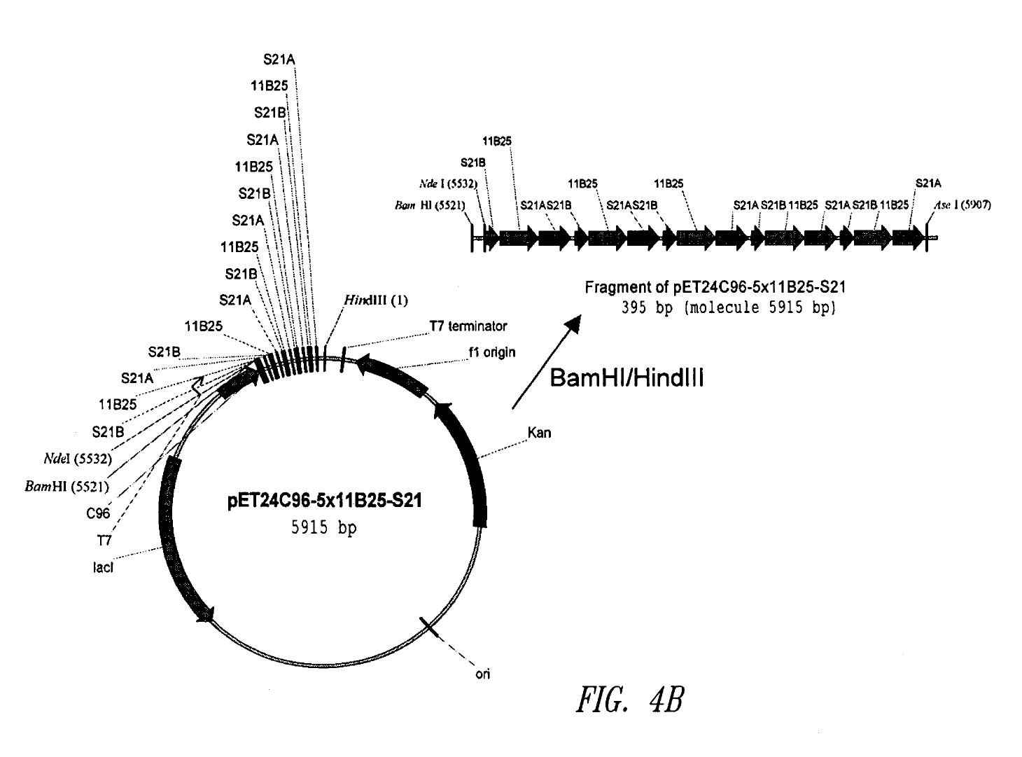

Finally, to facilitate expression testing, the constructs were transferred to an expression vector having a T7 promoter, such as pET24C96, using the restriction enzymes BamHI and HindIII (FIG. 4). The nucleic acid sequence of all of the aforementioned constructs was verified by sequencing.

### Example 3

### Temperature-Inducible Nucleic Acid Expression Vector

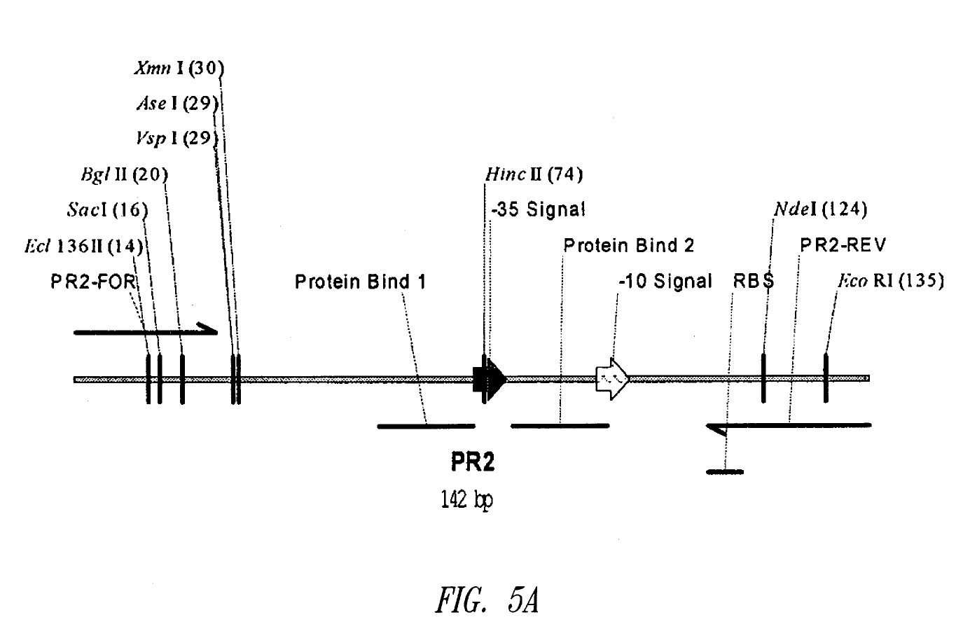

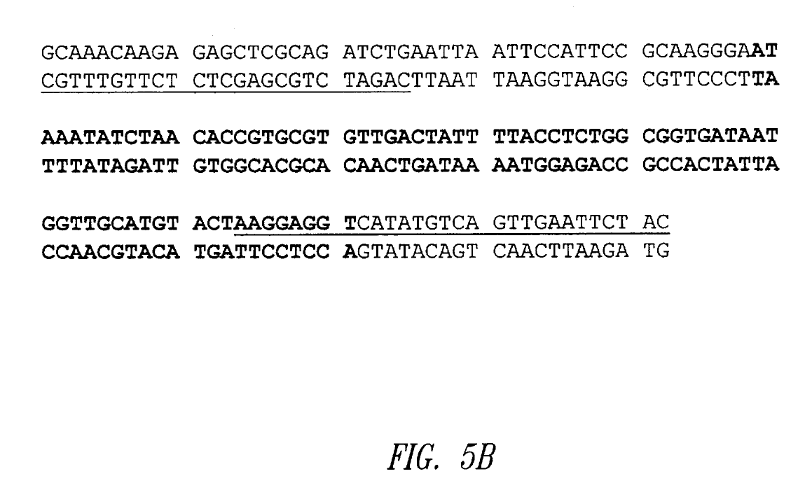

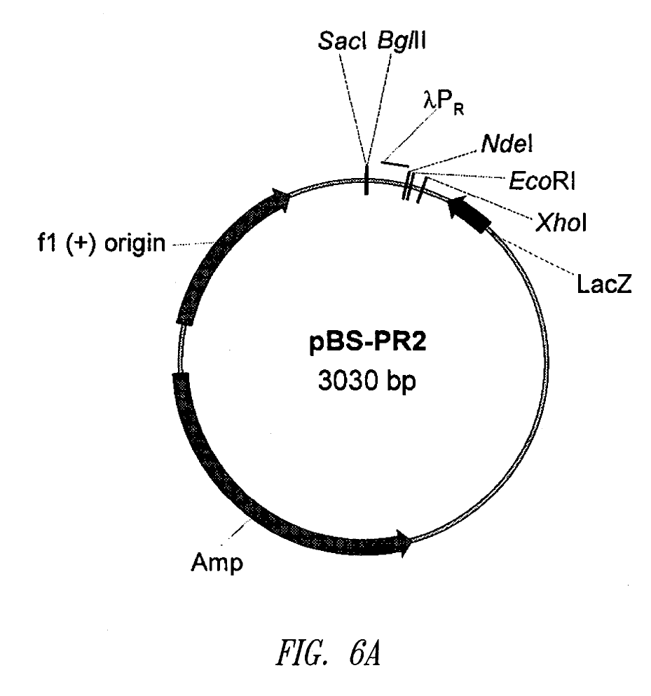

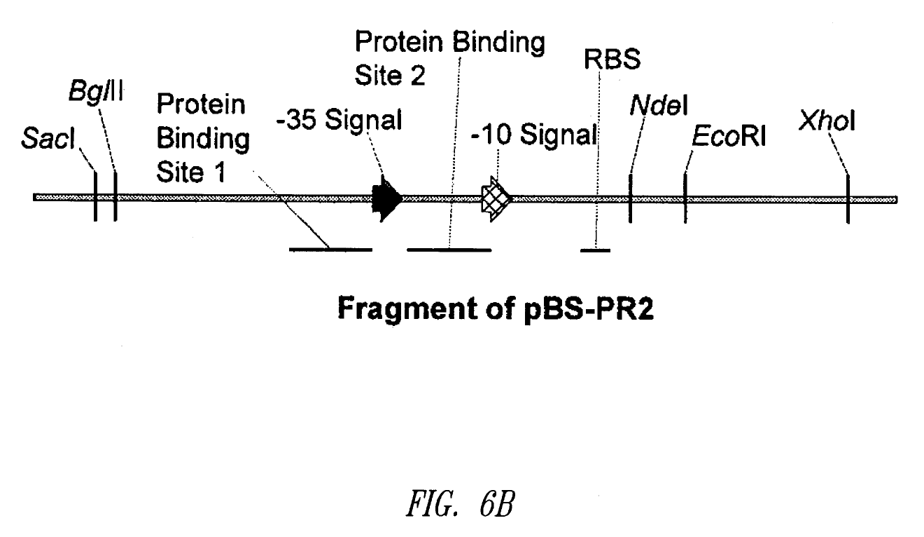

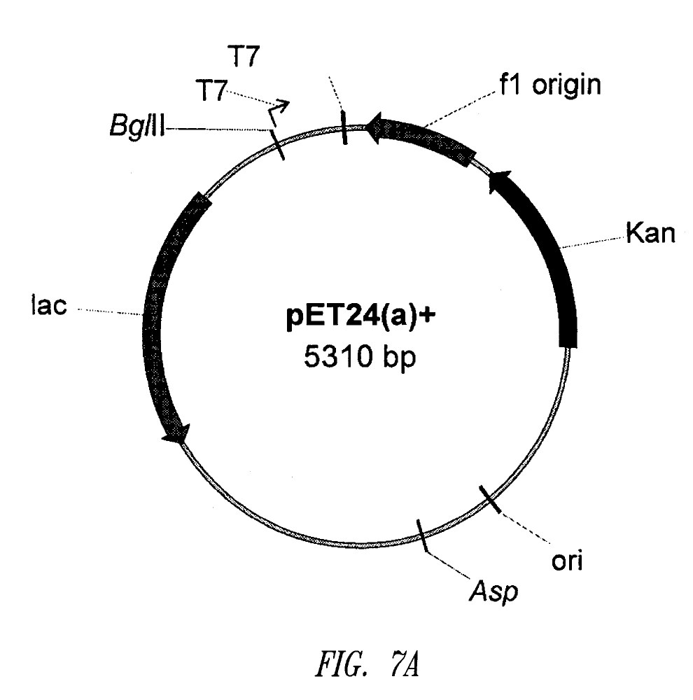

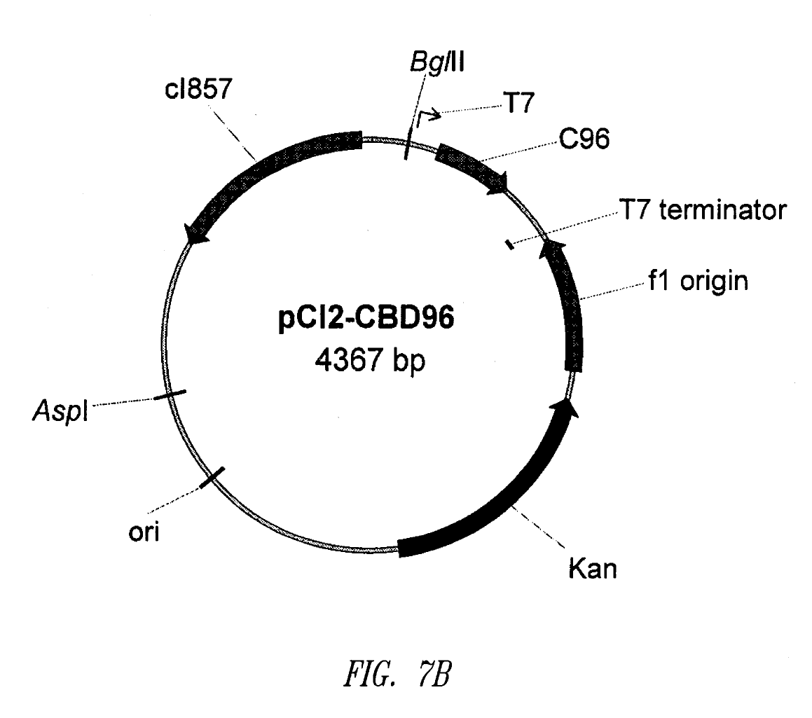

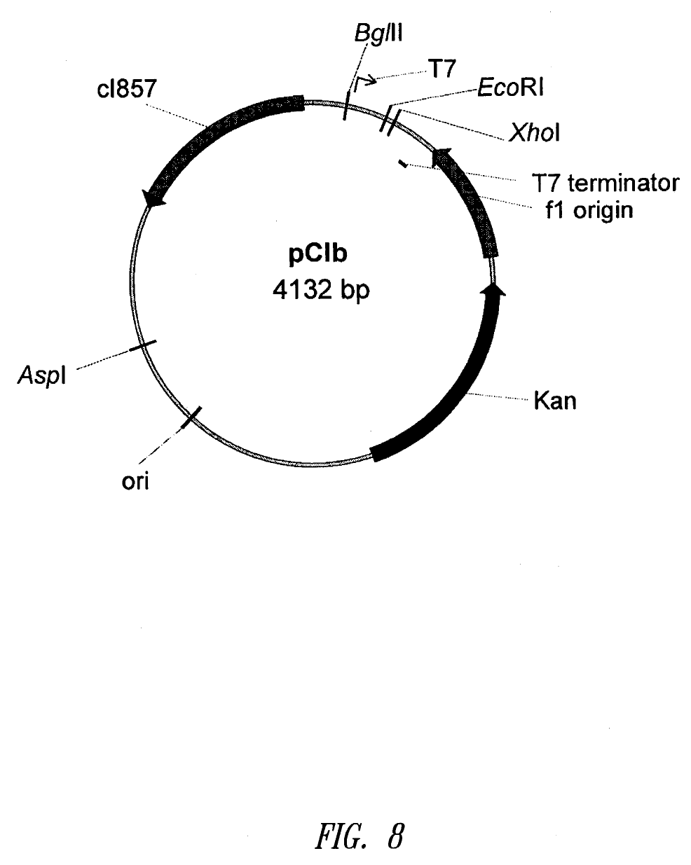

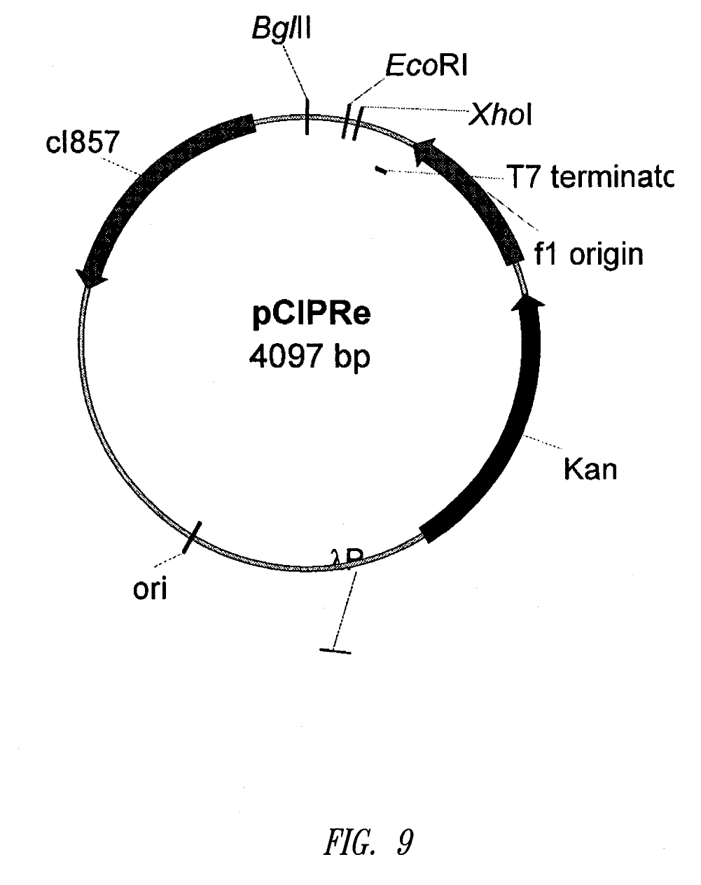

An alternative vector having a temperature sensitive promoter is used to generate heat inducible nucleic acid expression constructs to produce precursor cationic peptide fusion proteins. To this end, a nucleic acid expression vector is constructed comprising the λ P_{R} promoter and encoding the temperature sensitive λ repressor, cI857. The λ P_{R} promoter, which corresponds to nucleotides 37,965-38,037 of the λ phage chromosome, is synthesized as an oligonucleotide (PR2) having SacI and Bg/II restriction sites incorporated in the 5′ end, and NdeI and EcoRI restriction sites incorporated in the 3′ end (FIG. 5). The PR2 is PCR amplified with primers PR2-For and PR2-Rev and the resulting product can be cloned into pBluescript to create pBS-PR2 (FIG. 6). To prepare a vector containing the cI857 repressor, a Bg/II/AspI fragment containing the cI857 sequence from pCI2-CBD96 (FIG. 7) can be ligated into the corresponding sites of pET24a(+) (Novagen, Madison, Wis.) (FIG. 7) to obtain pClb (FIG. 8). The repressor sequence of pCI2-CBD96 can be obtained from pCG30 (ATCC 87698). Then a Bg/II/EcoRI fragment containing the λ P_{R} promoter is ligated into the corresponding sites in pClb to obtain pCIPRe (FIG. 9), to create a heat inducible nucleic acid expression vector having the λ P_{R} promoter and the cI857 repressor useful for the production of precursor cationic peptide/spacer fusion proteins.

### Example 4

### Expression of a Fusion Protein Containing a Precursor Cationic Peptide

Each of the nucleic acid expression constructs as described in Example 2 were transformed into Escherichia coli containing a genomic copy of the T7 RNA polymerase gene operably fused to the IPTG-inducible promoter lacUV5 (e.g., E. coli HMS174(DE3) or BL21(DE3)). Transformed E. coli were grown in Terrific Broth (TB) broth containing 1% dextrose and 30 μg/mL kanamycin, and in the presence or absence of IPTG. The TB broth is prepared as follows: 12 g trypticase peptone (BBL, Baltimore, Md.), 24 g yeast extract (BBL, Baltimore, Md.), and 4 mL glycerol (Fisher) is added to 900 mL of Milli-Q water. The broth is autoclaved at 121° C. for 20 minutes and 100 mL of autoclaved 0.17 M KH₂PO₄ (VWR, Buffalo Grove, Ill.), 0.72 M K₂HPO₄ (Fisher Scientific, Santa Clara, Calif.) is added, which results in a pH of 7.4.

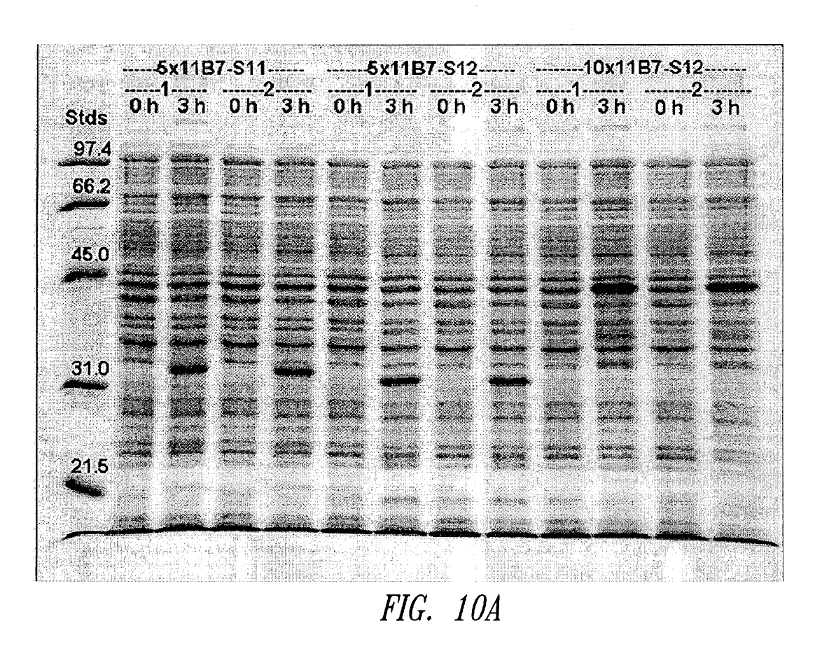

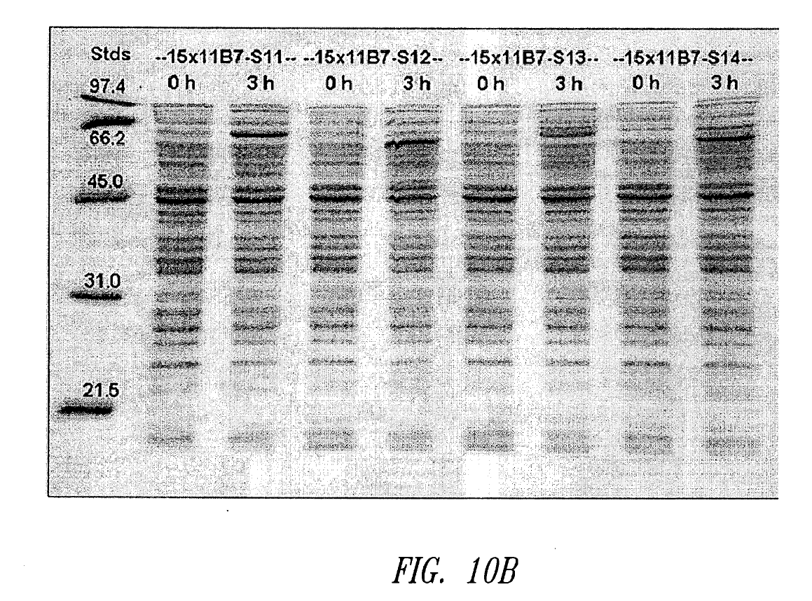

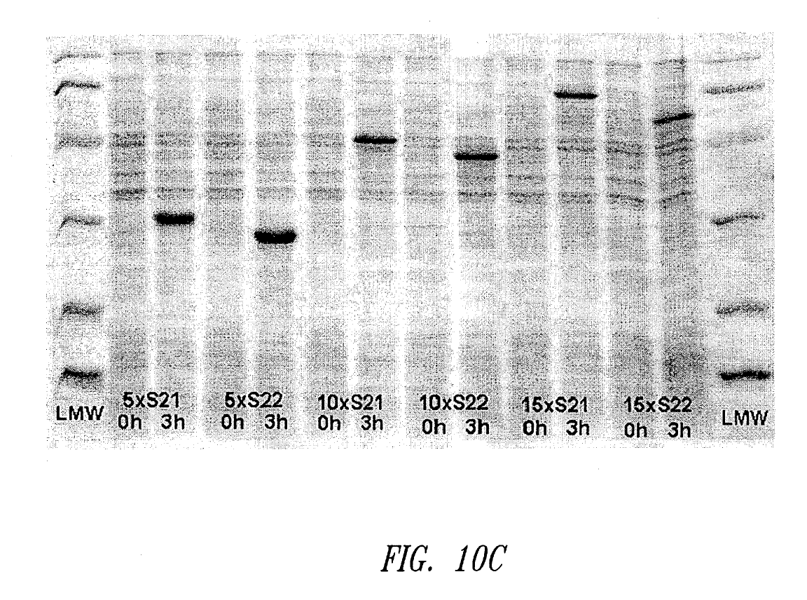

To analyze fusion protein expression on a small scale, cultures were grown in 3 mL of TB at 37° C. with vigorous shaking overnight, 1 mL of the overnight culture was diluted with 1 mL fresh TB and IPTG was added, followed by incubation of the cultures at 37° C. with vigorous shaking for another 3 hours. Most of the constructs having 5× copies of precursor indolicidin analogue/spacer showed a high level of expression, while more variable expression occurred with the 10× and 15× precursor indolicidin analogue/spacer constructs (FIG. 10 and Table 3). In most cases, expression levels were high enough to result in the production of inclusion bodies containing the expressed fusion protein. Frozen stocks of the recombinant E. coli of interest were prepared by standard methods known in the art and stored at −80° C.

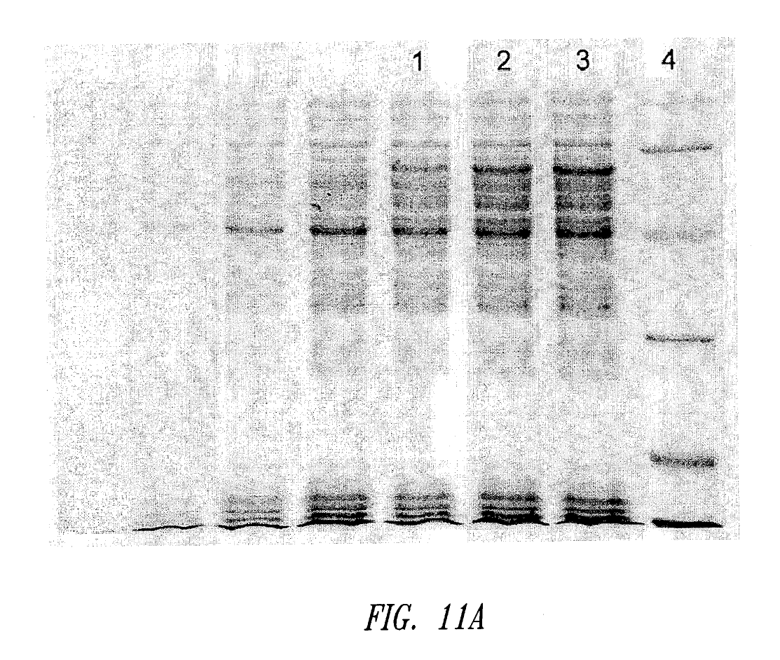

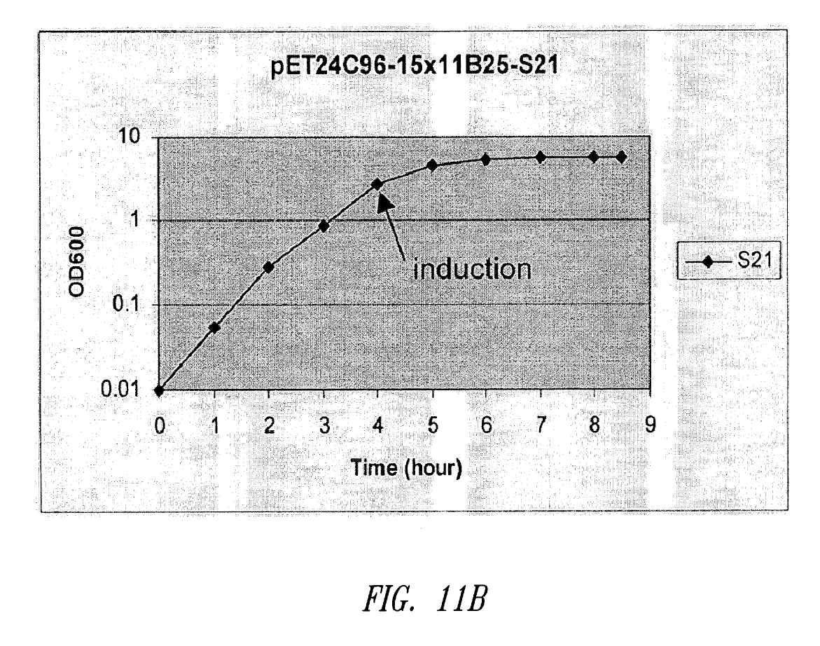

To prepare an inoculum for 1L cultures, cells from the frozen stocks were cultured 4-7 hours, aliquoted, and then kept frozen in 8% glycerol at −80° C. before use. A 1L culture was then inoculated with a 1% volume of frozen inoculum and grown for 5 hours before inducing expression with 0.5 mM IPTG. Samples were taken each hour and the proteins of whole-cell lysates were separated by SDS-PAGE (FIG. 11).

TABLE 3

Expression Levels of Various indolicidin
analogue-Spacer Fusions

% Expression*

| | | | |
|---|---|---|---|
| Spacer | Sequence (11B7-spacer) | 5x | 15x |

| | | | |
|---|---|---|---|
| S11 | ILRWPWWPWRRK-AEAEPEAEAEGK | 21 | 15 |
| S12 | ILRWPWWPWRRK-AEAEPEAEAAGK | 23 | 12 |
| S13 | ILRWPWWPWRRK-AEAEPEAEAEGPK | 15 | 7.5 |
| S14 | ILRWPWWPWRRK-AEAEPEAEAAGPK | 15 | 11 |
| S19 | ILRWPWWPWRRK-AEAEPELAEAAGK | 23 | 9.1 |
| S20 | ILRWPWWPWRRK-AEAEPELVEAAGK | 26 | 12 |
| S29 | ILRWPWWPWRRK-MAEAEPEAEPIMK | 12 | 11 |
| S30 | ILRWPWWPWRRK-MAEAEPEAEEPIMK | 23 | 3.6 |
| S31 | ILRWPWWPWRRK-MAEAEPEAEAPIMK | 10 | NA |
| S32 | ILRWPWWPWRRK-MAEAEPEAEPIMEK | 20 | 3.1 |
| S33 | ILRWPWWPWRRK-MAEAEPEAEPIMVK | 6 | Not Tested |
| S34 | ILRWPWWPWRRK-MAEAEPEAEEPIMK | 24 | 3.0 |
| S35 | ILRWPWWPWRRK-MAEAEPEAEAPIMK | 11 | Not Tested |

*% fusion protein/total cell protein

### Example 5

### Solubilization of Fusion Protein in Inclusion Bodies

To isolate the expressed fusion proteins, cells were harvested by centrifugation three to five hours after induction, lysed by suspension in 200 mL 50 mM Tris, 10 mM EDTA pH 8.0, sonicated, and treated with lysozyme (100 μg/mL, room temperature, 30 min). Inclusion bodies were then isolated by centrifugation at 22,000×g for 30 min at 4° C., followed by washes of the resulting pellet in 200 mL 1% Triton X-100@, 100 mM NaCl, and Milli-Q water.

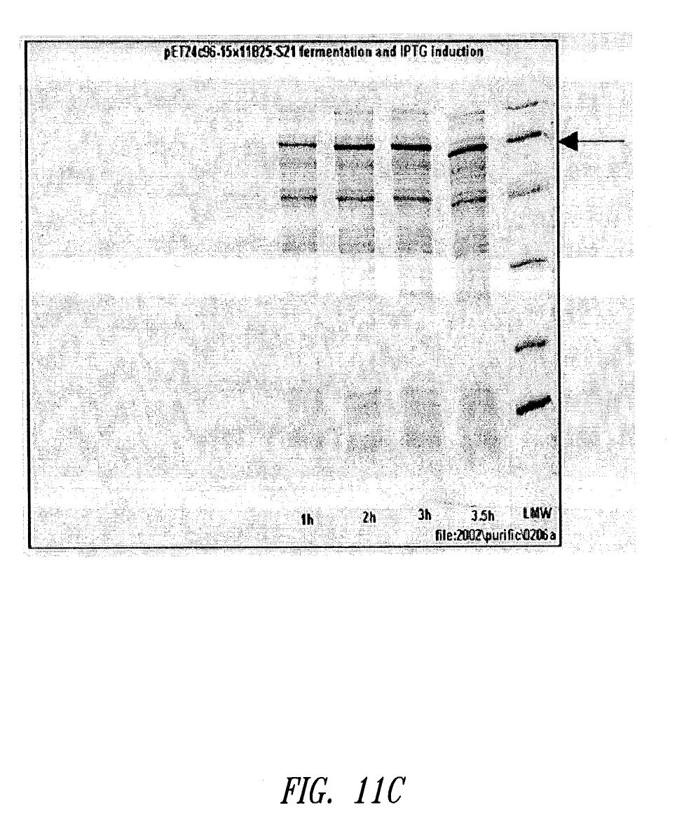

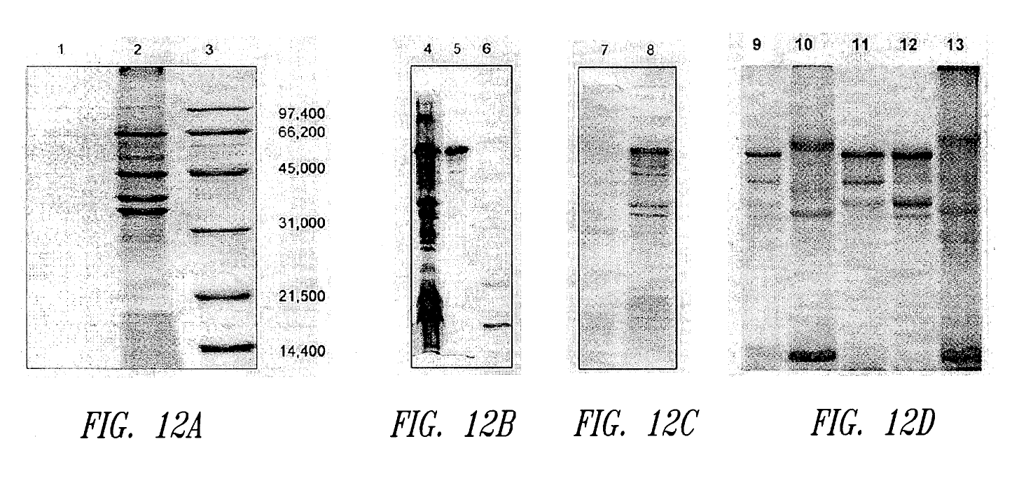

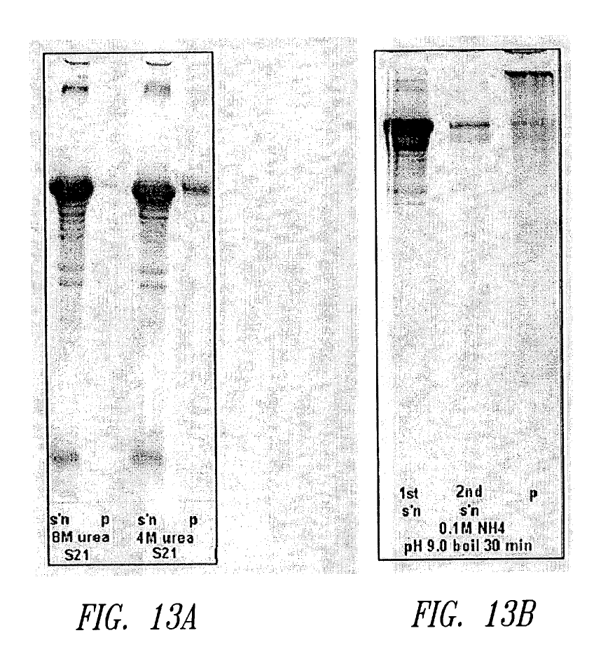

For efficient enzymatic cleavage of the precursor indolicidin analogue/spacer fusion protein accumulated in inclusion bodies, the protein should be solubilized. A variety of solubilization reagents are typically used in the art, including guanidine hydrochloride, SDS, and urea. Therefore, the fusion proteins were initially solubilized in urea (FIGS. 12A, 12B, 12D, and 13A) and SDS (FIG. 12C).

However, many cleaving agents are incompatible with high urea concentrations. Thus, the urea concentration was reduced by either an 8× dilution of the preparation or by a buffer exchange to obtain conditions compatible with enzymatic cleavage. The fusion proteins were cleaved by lysyl endopeptidase, but the cleavage was inefficient (see FIG. 13A), even at reduced (e.g., 1.0 M) urea concentration. In addition, the presence of urea was a complication for the purification steps described herein.

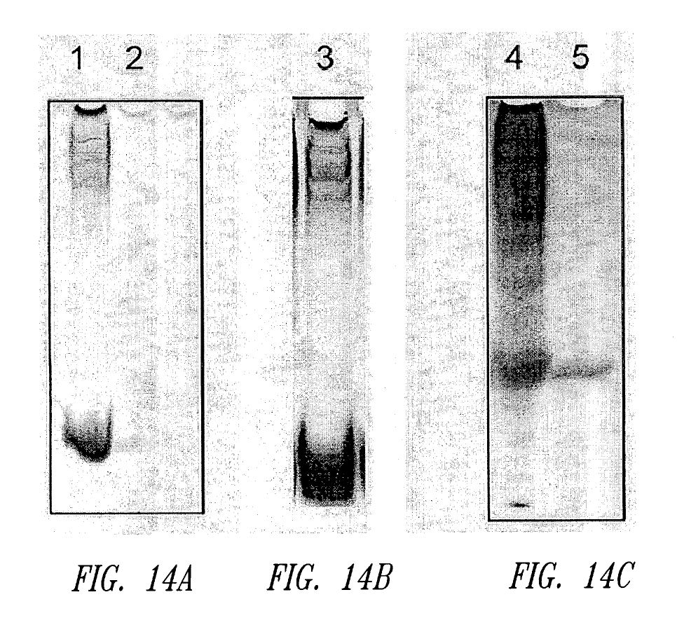

As an alternative, the fusion proteins were solubilized with ammonium bicarbonate (NH₄HCO₃, 0.1 M). Surprisingly, the NH₄HCO₃ functioned efficiently as a solubilization agent (FIG. 12D) and lysyl endopeptidase showed high efficiency when cleaving fusion proteins in a NH₄HCO₃ buffer (see FIG. 14 and Example 6). Use of NH₄HCO₃ as the solubilization agent had several advantages including: (a) no requirement for an additional dilution step or buffer exchange, (b) cleavage was very efficient with a high yield of the resulting precursor indolicidin analogue (e.g., 11B7), and (c) the resulting peptide preparation showed less impurities as compared to solubilization with urea.

### Example 6

### Lysyl Endopeptidase as Cleaving Agent for Fusion Proteins

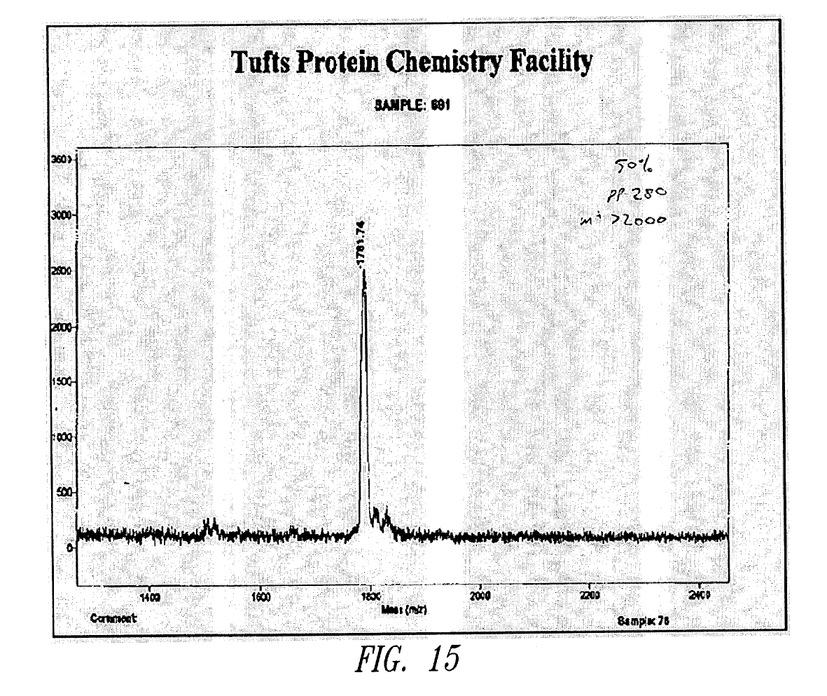

The solubilized fusion protein may then be enzymatically (or chemically) cleaved with a variety of enzymes. One limitation of chemical cleavage is that the released peptide may no longer be a free acid (e.g., cyanogen bromide results in a peptide having a carboxy-terminal homoserine/homoserine lactone). On the other hand, enzymes often require a specific amino acid or sequence for cleavage to occur. For example, lysyl endopeptidase requires a Lys (K) for enzymatic cleavage. In one embodiment, an indolicidin analogue was successfully released from the fusion protein upon cleavage with lysyl endopeptidase at lysyl endopeptidase:fusion protein ratios of 1:20, 1:50 and 1:100 (FIG. 14). The identity of the 11B7 peptide was confirmed by mass spectrometry (FIG. 15), as well as by sequence analysis. Unknown, however, was whether a particular amino acid sequence adjacent to the Lys would alter lysyl endopeptidase activity.

To identify the best spacer sequence at the spacer-indolicidin analogue junction, different amino acid sequences were placed next to the Lys cleavage site. Of particular interest was the potential inhibitory effect of negatively charged amino acids, such as Glu (E), because the spacers used in this invention contain such negatively charged amino acids to compensate for the positive charge of the indolicidin analogue. To evaluate the effect on cleavage rate of having the amino acid Glu (E) close to the cleavage site, “model” peptides were used as substrates for lysyl endopeptidase. The “model” peptides contain a portion of a spacer (6-7 amino acids) and a portion of the precursor indolicidin peptide (6 amino acids), with a lysyl endopeptidase cleavage site connecting the two portions. The results indicate that a negatively charged amino acid (e.g., glutamic acid, E) hinders cleavage at position −3 and even at position −4. Furthermore, the addition of a proline (P) in position −1 facilitated cleavage. Hence, negatively charged amino acids close to the cleavage site, in particular at positions −3 and −4, alter lysyl endopeptidase activity. In preferable embodiments, the spacers have neutral or hydrophobic amino acids within the first 1 to 5 amino acids of the cleavage site. Based on these findings, full-length fusion proteins having modified cleavage sites were tested for cleavage with lysyl endopeptidase (Table 4).

TABLE 4

uz,1/32 Fusion Proteins with Modified Cleavage Sequences

| | |
|---|---|
| Expres- | Cleav- |
| sion | age |

| | | |
|---|---|---|
| Peptide- | ( %*) | effi- |

| | | | | |
|---|---|---|---|---|
| spacer | Peptide-Spacer Sequence | 5x | 15x | ciency |

| | | | | |
|---|---|---|---|---|
| 11B7-S11 | ILRWPWWPWRRK-AEAEPEAEAEGK | 21 | 15 | −/+ |
| 11B7-S12 | ILRWPWWPWRRK-AEAEPEAEAAGK | 23 | 12 | ++++ |
| 11B7-S19 | ILRWPWWPWRRK-AEAEPELAEAAGK | 23 | 9.1 | +++ |
| 11B7-S20 | ILRWPWWPWRRK-AEAEPELVEAAGK | 26 | 12 | ++++ |
| 11B7-S13 | ILRWPWWPWRRK-AEAEPEAEAEGPK | 15 | 7.5 | Not |
| | | | | Tested |
| 11B7-S14 | ILRWPWWPWRRK-AEAEPEAEAAGPK | 15 | 11 | Not |
| | | | | Tested |
| 11B7-S30 | ILRWPWWPWRRK-MAEAEPEAEEPIMK | 23 | 3.6 | ++++ |

*% fusion protein/total cell protein

The cleavage results for the full-length fusion proteins correlate with the model peptide results.

### Example 7

### Kexin as Cleaving Agent for Fusion Proteins

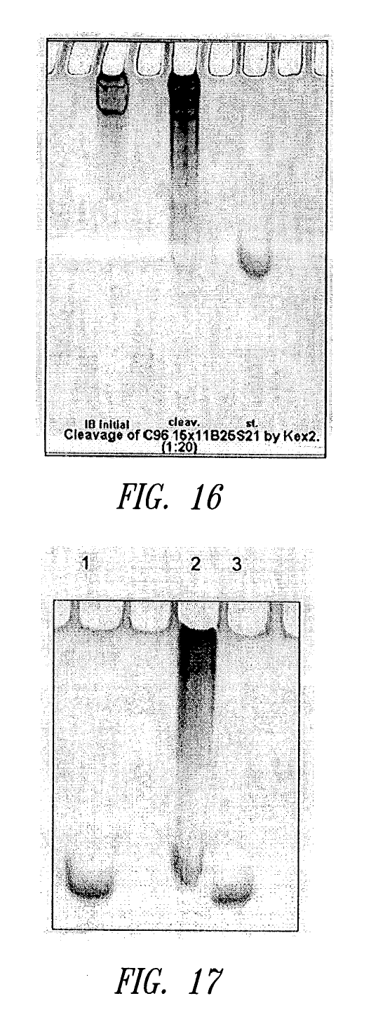

While precursor cationic peptide was successfully released from the fusion protein upon cleavage with lysyl endopeptidase (FIG. 14), surprisingly the cleavage with the soluble form of yeast kexin (Kex2) (see, e.g., Brenner and Fuller, Proc Nat'l Acad Sci USA 89:922, 1992) was inefficient at kexin:fusion protein ratios of 1:20 and 1:100 (FIG. 16). Model peptides were synthesized and tested for cleavage with Kex2. Surprisingly, and in contrast to lysyl endopeptidase, only the constructs having a Peptide-Spacer structure were cleaved, while constructs having the Spacer-Peptide structure were not cleaved, even when the enzyme:substrate ratio was 1:500. In addition, non-specific cleavage was observed when using this high enzyme concentration. Further model peptides with variations in the amino acids preceding the cleavage site showed no improvement of Kex2 cleavage. Thus, Kex2 cleavage cannot be used for the efficient production of the precursor indolicidin peptides of the instant invention.

### Example 8

### Purification of Released Precursor Cationic Peptide

Following the enzymatic cleavage of the fusion protein, the released precursor cationic peptide (i.e., precursor indolicidin analogue) can be purified using a combination of chromatographic and filtration methods. For example, purification may be accomplished by using anion exchange chromatography (Macro-Prep High Q Support, Bio-Rad Laboratories, Hercules, Calif.) and/or reverse phase chromatography (Poros 50 R2 Resin, PerSeptive Biosystems). The purification of the precursor indolicidin analogue 11B7 peptide was performed on a BioSys™ 2000 chromatography work station (Beckman Instruments, Inc.), using 1 mL Fast Flow Q-Sepharose anion exchange resin (Pharmacia Biotech AB) packed in an HR column (1×5 cm). The column was equilibrated with 5 column volumes (CV) of 0.5M NaOH at a flow rate of 5 mL/min, followed by a water wash. Monitored at 280 nm were conductivity, pH, and absorbency. When the conductivity dropped down to 0 mS, the cleaved mixture in 0.1 M of ammonium bicarbonate pH 9 was applied onto the column. The unbound pure cationic peptide flowed through the column and was monitored as the leading peak. When the absorbance dropped to baseline, the bound material (i.e., impurities) was washed off the column with 0.5 M NaOH and appeared as the second peak.

The flow-through peak was collected and pooled and the pH was adjusted to 7.0-7.5 with 1 M HCl. The sample was analyzed for purity by reverse phase HPLC, using a C8 column (4.6×10, Nova-Pak, Waters) and by acid-urea polyacrylamide gel electrophoresis (West and Bonner, Biochemistry 19:3238, 1980). The peptide purification is shown on an acid-urea polyacrylamide gel (FIG. 17).

### EXAMPLE 9

### Amidation of the Purified Precursor Cationic Peptide Carboxylic Acid

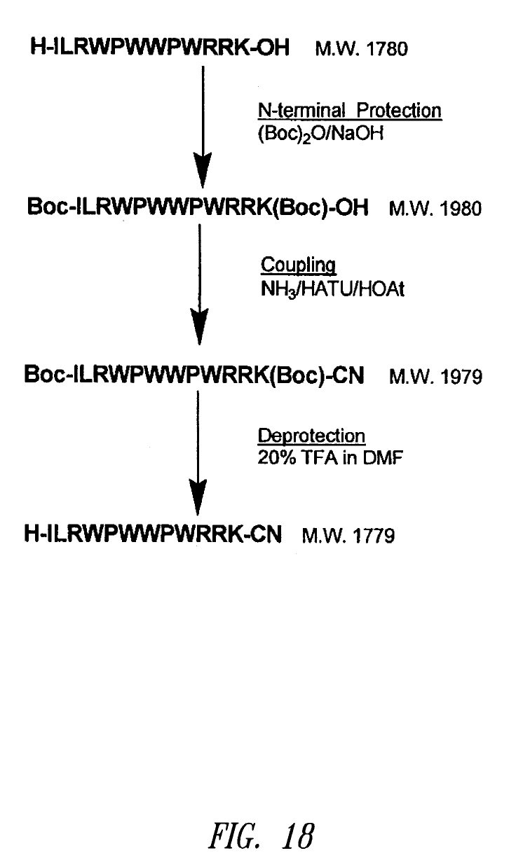

When the precursor anti-infective peptide that is to be modified is full length, then the final step in producing a modified recombinant cationic peptide and analogue or derivative thereof is to amidate the purified precursor cationic peptide carboxylic acid. In this embodiment, a precursor indolicidin 11B7 analogue (ILRWPWWPWRRK) is converted to the desired 11B7CN indolicidin analogue (ILRWPWWPWRRK-CN) (i.e., carboxy-terminal amidated 11B7). The following steps were performed to couple the recombinant 11B7 precursor indolicidin analogue with ammonia in the presence of coupling reagent to generate 11B7CN (FIG. 18).

As an initial reaction, the 11B7 precursor indolicidin analogue was bocylated as follows. Di-tert-butyl dicarbonate (40 mg, 0.18 mmol) was added as a solid to a solution of 11B7 precursor indolicidin analogue (53 mg, 0.03 mmol) in acetonitrile (5.0 mL), 1 N NaOH (1.0 mL), and H₂O (5.0 mL). The reaction mixture was stirred at room temperature and the solvents were removed under vacuum. The residue was redissolved in H₂O and extracted with n-hexane. The aqueous layers were combined, diluted with H₂O, and lyophilized to obtain 52 mg of white powder (yield 87%). The product obtained (Di-Boc-11B7-OH; MS: m/z 1980) was used without further purification.

Then HATU (O-(1H-9-azabenzotriazole-1-yl)-1,1,3,3-tetramethyluronium hexafluorophosphate; 23 mg, 0.06 mmol) and HOAt (N-hydroxy-9-azabenzotriazole; 8.2 mg, 0.06 mmol) were added to a solution of Di-Boc-11B7-OH (20 mg, 0.01 mmol) and diisopropylethylamine (DIEA; 0.035 mL, 0.2 mmol) in 6.0 mL dimethylformamide (DMF), followed by the addition of ammonia in methanol (2.0 M, 0.15 mL, 0.30 mmol). The reaction mixture was stirred at room temperature for 3 hours and the crude product was checked by matrix-assisted laser desorption ionization-time of flight (MALDI-TOF). The molecular mass of the product obtained (m/z 1979) corresponds to the expected product, Di-Boc-11B7-CN. To deprotect the resulting peptide, 0.2 mL trifluoroacetic acid (TFA) was added to a solution of crude Di-Boc-11B7-CN in 0.8 mL of DMF and the reaction mixture was stirred for 30 minutes at room temperature.

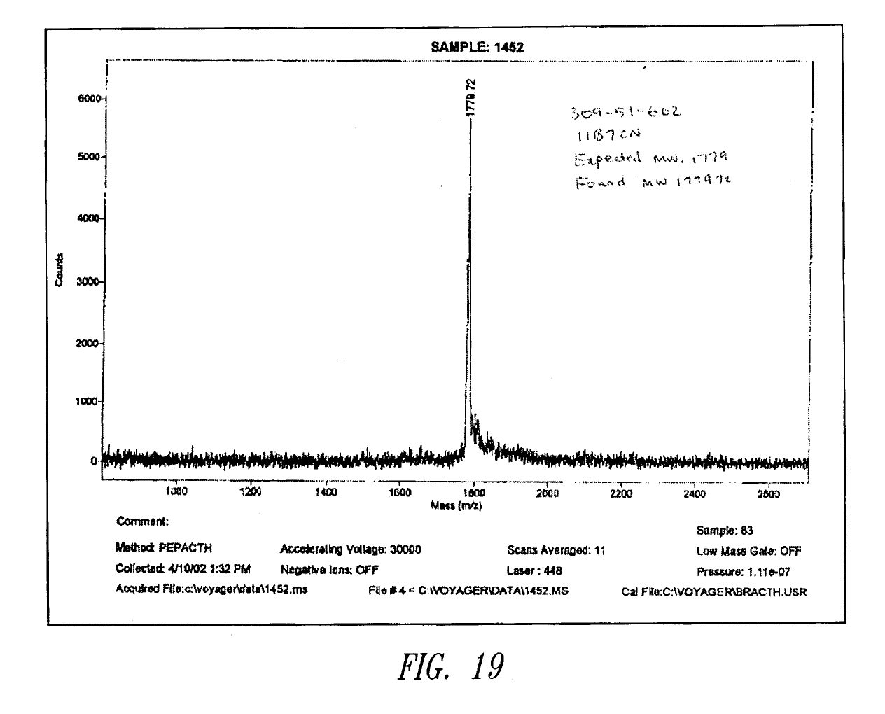

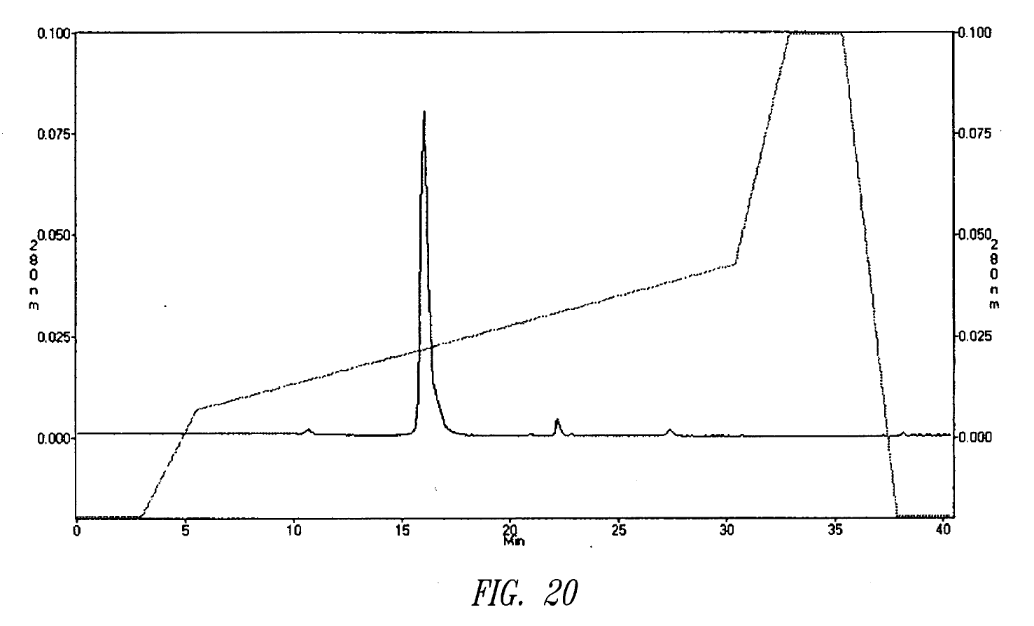

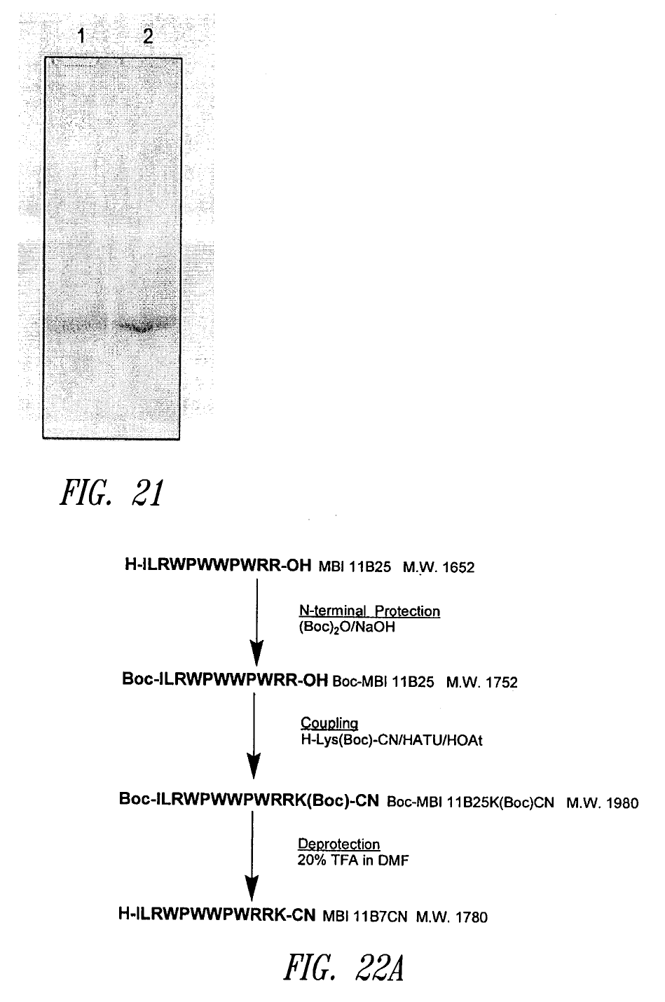

The product was analyzed by MALDI-TOF and showed a major peak of m/z 1779, which corresponds to the molecular mass of the desired product, the amidated indolicidin analogue 11B7CN (FIG. 19). The crude product was purified by a semi-preparative Reverse Phase-HPLC and the majorfraction was lyophilized to give 8.3 mg of purified peptide 11B7CN (FIG. 20; two-step yield 47%). The migration profile was identical to the standard 11B7CN on an acid-urea polyacrylamide gel (FIG. 21).

### Example 10

### Modification of the Purified Precursor Cationic Peptide

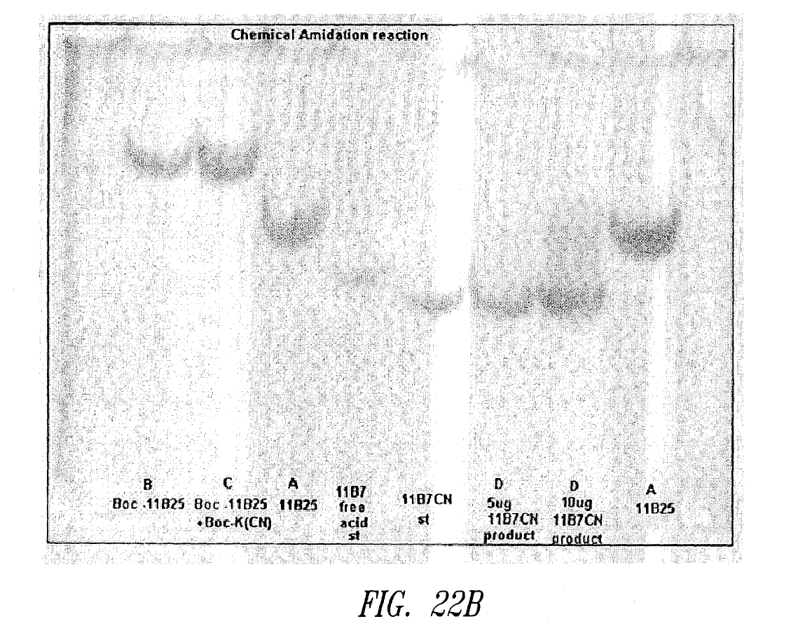

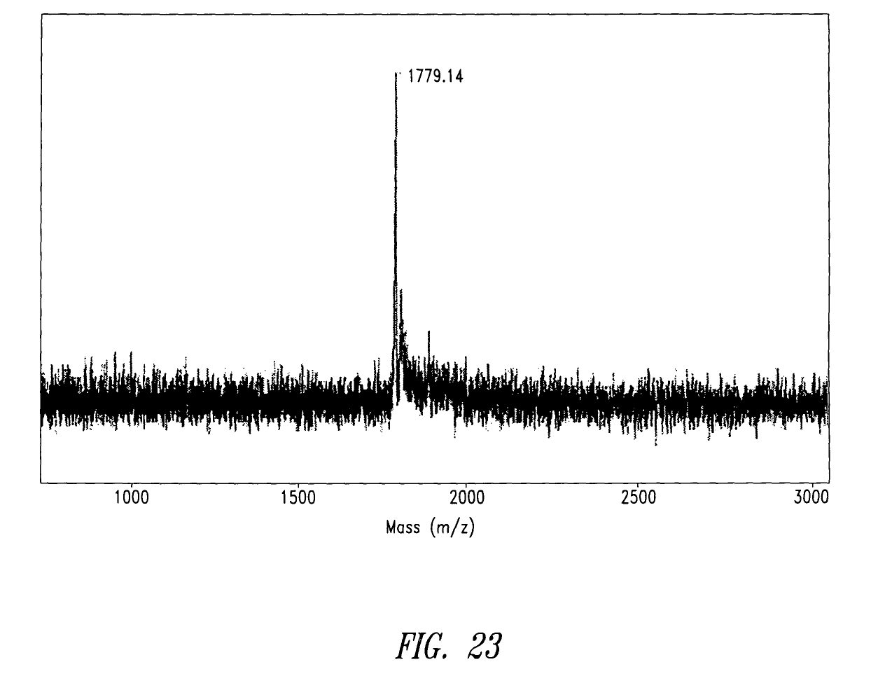

When the precursor anti-infective peptide that is to be modified is less than full length, then the final step in producing a recombinant cationic peptide and analogue or derivative thereof is to chemically couple one or more amino acids that comprise at least one non-natural amino acid, such as an amidated amino acid or a D-amino acid, to the purified precursor cationic peptide. In this embodiment, a precursor indolicidin 11B25 analogue (ILRWPWWPWRR) is coupled to a lysine amide (K-CN) to generate a desired 11B7CN indolicidin analogue (ILRWPWWPWRRK-CN) (i.e., carboxy-terminal amidated 11B7). The following steps were performed to couple the recombinant 11B25 precursor indolicidin analogue with K-CN to generate 11B7CN (FIG. 22).

As an initial reaction, the 11B25 precursor indolicidin analogue is bocylated as follows. Di-tert-butyl dicarbonate (10 mg, 0.0458 mmol) was added as a solid to a solution of 11B25 precursor indolicidin analogue (19 mg, 0.0115 mmol) in acetonitrile (3.0 mL), 1 N NaOH (0.5 mL), and H₂O (4.0 mL). The reaction mixture was stirred at room temperature and the solvents were removed under vacuum. The residue was redissolved in H₂O and extracted with n-hexane. The aqueous layers were combined, diluted with H₂O, and lyophilized to obtain 23 mg of white powder. The product obtained (Boc-11B25; MS: m/z 1753) was used without further purification.

Then HATU (O-(1H-9-azabenzotriazole-1-yl)-1,1,3,3-tetramethyluronium hexafluorophosphate; 2.8 mg, 0.0072 mmol) and HOAt (N-hydroxy-9-azabenzotriazole; 1.0 mg, 0.0072 mmol) are added to a solution of Boc-11B25 (4.2 mg, 0.0024 mmol) and H-Lys(Boc)-NH₂ (2.1 mg, 0.0072 mmol) in 1.2 mL dimethylformamide (DMF), followed by the addition of diisopropylethylamine (DIEA; 0.013 ml, 0.072 mmol). The reaction mixture was stirred at room temperature for 3 hours and the crude product was checked by matrix assisted laser desorption ionization-time of flight (MALDI-TOF). The molecular mass of the product obtained (m/z 1980) corresponds to the expected product, Boc-11B7-K13(ε-N-Boc)-CN. To deprotect the resulting peptide, 0.2 ml trifluoroacetic acid (TFA) was added to a solution of crude Boc-11B7-K13(ε-N-Boc)-CN in 0.8 mL DMF and the reaction mixture was stirred for 30 minutes at room temperature.

The product was analyzed by MALDI-TOF and showed a major peak of m/z 1780, which corresponds to the molecular mass of the desired product, the amidated indolicidin analogue 11B7CN (FIG. 23).

From the foregoing it will be appreciated that, although specific embodiments of the invention have been described herein for purposes of illustration, various modifications may be made without deviating from the spirit and scope of the invention.

1. A method for producing a modified anti-infective cationic peptide, comprising:
expressing a fusion protein from a nucleic acid expression construct having an expression control element operably linked to a nucleic acid encoding a fusion protein comprising a precursor cationic peptide fused to an anionic spacer, wherein the fusion protein has the structure [(cationic peptide)(cleavage site)(anionic spacer)(cleavage site)]ₙ, wherein n is 5-10;
contacting the fusion protein with a cleaving agent to release the precursor cationic peptide from the anionic spacer, wherein the cleaving agent is lysyl endopeptidase;
isolating the precursor cationic peptide from the anionic spacer; and
contacting the isolated precursor cationic peptide with at least one amino acid under conditions and for a time sufficient to couple the precursor peptide with said at least one amino acid, wherein said at least one amino acid is a non-natural amino acid, and thereby producing a modified anti-infective cationic peptide.

2. The method according to claim 1 wherein the precursor cationic peptide is indolicidin analogue 11B25.

3. The method according to claim 1 wherein the anionic spacer is selected from the group consisting of AEAEPEAEAEGK, AEAEPEAEAAGK, AEAEPEAEAEGPK, AEAEPEAEAAGPK, AEAEPELAEAAGK, AEAEPELVEAAGK, MAEAEPEAEPIMK, MAEAEPEAEEPIMK, MAEAEPEAEAPIMK, MAEAEPEAEPIMEK, or MAEAEPEAEPIMVK.

4. The method according to any one of claims 1-3 wherein the non-natural amino acid is an amidated natural amino acid.

5. The method according to claim 4 wherein the amidated natural amino acid is lysine.

6. The method according to any one of claims 1-3 wherein the modified cationic peptide is indolicidin analogue 11B7CN.

7. A method for producing an amidated anti-infective cationic peptide, comprising:
expressing a fusion protein from a nucleic acid expression construct having an expression control element operably linked to a nucleic acid encoding a fusion protein comprising a precursor cationic peptide fused to an anionic spacer, wherein the fusion protein has the structure [(cationic peptide)(cleavage site)(anionic spacer)(cleavage site)]ₙ, wherein n is 5-10 and the fusion protein is expressed as an inclusion body;
solubilizing the fusion protein with ammonium carbonate;
contacting the fusion protein with a cleaving agent to release the precursor cationic peptide from the anionic spacer;
isolating the precursor cationic peptide from the anionic spacer; and
amidating the isolated precursor cationic peptide, and thereby producing an amidated anti-infective cationic peptide.

8. The method according to claim 7 wherein the cleaving agent is lysyl endopeptidase.

9. The method according to claim 7 wherein the anionic spacer is selected from the group consisting of AEAEPEAEAEGK, AEAEPEAEAAGK, AEAEPEAEAEGPK, AEAEPEAEAAGPK, AEAEPELAEAAGK, AEAEPELVEAAGK, MAEAEPEAEPIMK, MAEAEPEAEEPIMK, MAEAEPEAEAPIMK, MAEAEPEAEPIMEK, or MAEAEPEAEPIMVK.

10. The method according to claim 7 wherein the ammonium carbonate is at a concentration ranging from about 50 mM to about 250 mM.

11. The method according to any one of claims 7-10 wherein the amidated cationic peptide is indolicidin analogue 11B7CN.
